SEQUENCE LISTING (12 sequences)

SEQ ID NO: 1 - DNA, 1830 bp, Homo sapiens
agctgtcggactcaccgggggccctaagcagcgagacctgaggccagacggaactacaacatgtaccatccacgagaattgtacccgtccctgggggccggctaccgcctggggcccgcccaacctggggccgactccagcttcccacccgccctagcggagggctaccgctaccccgaactggacacccctaaactggattgcttcctctccgggatggaggctgctccccgcaccctggccgcgcacccacctctgccccttctgccccctgccatgggcactgagccggccccatcagctccagaggccctccattccctcccgggggtcagcctgagcctggagaaccgggagctatggaaggagttcagctctgtgggaacagaaatgatcatcaccaaagctgggaggcgcatgttccctgcctgccgagtgtcagtcactggcctggaccccgaggcccgctacttgtttcttctggatgtgattccggtggatggggctcgctaccgctggcagggccggcgctgggagcccagcggcaaggcagagccccgcctgcctgaccgtgtctacattcaccccgactctcctgccactggtgcacattggatgcggcagcctgtgtctttccatcgtgtcaagctcaccaacagcacgctggacccccacggccacctgatcctgcactccatgcacaagtaccaaccccgcatacacctagttcgggcagcccagctctgcagccagcactgggggggcatggcctccttccgcttccccgagaccacattcatctccgtgacagcctaccagaacccacagatcacacaactgaagattgcagccaatccctttgccaaaggcttccgggagaacggcagaaactgtaagagggagcgagacgcccgtgtgaagaggaaactgcggggcccagagccagcagccacagaggcctatgggagcggagacacaccaggtggtccctgcgactccaccctgggtggagacattcgtgaatcagatccagaacaggccccagcccccggggaagccaccgctgccccggcacctctgtgtggtggccccagtgctgaggcctacctcctgcaccctgcggctttccatggggcccccagtcaccttcccaccaggagccccagcttcccggaggctccagactccgggcgctcagccccctactcggctgcatttctggagctgccgcacgggtcagggggctccgggtacccagcggctccaccggcggtaccctttgccccgcactttctccaagggggccccttccctctaccatacaccgcgcctgggggctatctggatgtgggctccaaacccatgtactgaaccactgctgggcccctctgcctccatcacaaacctcccgctcccttcccccagccctggagccccctcacctccaccggccccatcccccacaccaaatggccgccttgggcctgcccacccaccccctcaacttcacaccttgatttcactcccacccccctctggcttcaaagcctaaccaaggctgctgggagtccagctggggccggcttccccttcccggctctcacctccgtgtgaatggaaggggctctgcctggccaaatggggccacagaccagcatccccacttaccaggcctccccatgggctctggaggggctcaatccccacccccccacacacactggtgcaggccacaccagtctgttgttctgggaccagagtatttttgttaataaaactcaaaagccatccatgctcaggaaaaaaaaaaaaaaaaaaaaaaaaaaaaaa

SEQ ID NO: 2 - protein, 436 aa, Homo sapiens
MYHPRELYPSLGAGYRLGPAQPGADSSFPPALAEGYRYPELDTPKLDCFLSGMEAAPRTLAAHPPLPLLPPAMGTEPAPSAPEALHSLPGVSLSLENRELWKEFSSVGTEMIITKAGRRMFPACRVSVTGLDPEARYLFLLDVIPVDGARYRWQGRRWEPSGKAEPRLPDRVYIHPDSPATGAHWMRQPVSFHRVKLTNSTLDPHGHLILHSMHKYQPRIHLVRAAQLCSQHWGGMASFRFPETTFISVTAYQNPQITQLKIAANPFAKGFRENGRNCKRERDARVKRKLRGPEPAATEAYGSGDTPGGPCDSTLGGDIRESDPEQAPAPGEATAAPAPLCGGPSAEAYLLHPAAFHGAPSHLPTRSPSFPEAPDSGRSAPYSAAFLELPHGSGGSGYPAAPPAVPFAPHFLQGGPFPLPYTAPGGYLDVGSKPMY

SEQ ID NO: 3 - DNA, 1731 bp, Mus musculus
cacaaggccagaagaaactacaacatgtaccatccacgagagttgtacccctccctggggactggctaccgtctgggacacccccagcctggggcagactccaccttcccacctgccctgacagagggttaccgctaccctgatttggatacttctaaactggattgcttcctctctgggatcgaggcagctccccacactctggctgcagccgctcctttgccccttctcccatctgctctgggccccgagacagcaccgccacccccagaggcccttcactcgcttcctggggtcagcctgagcttggagaaccaggaactgtggaaggaattcagcgctgtggggacagagatgatcatcaccaaggctggcaggcgcatgttccctgcttgccgagtatcagtcactggcctggacccagaggcccgctacttgtttcttctggatgtggttccagtggatggggcccgataccgctggcagggccagcactgggagccaagtggcaaggctgaaccccgcctacccgaccgtgtctacattcaccctgactctcctgccactggtgcccactggatgcggcagcccgtatccttccatcgtgttaagctcaccaacagcacactggacccccatggccacctgatcttgcactcgatgcacaagtaccagcctcgcatccacctggtgagagccacccaactatgcagccaacactgggggggtgtggcctccttccgatttcctgagaccacattcatctctgtgacagcctaccagaaccctaggatcacacagctgaagatcgcagccaatccctttgccaaaggtttccgagaaaatggcagaaactgtaagagggagcgggatgcccgtgtgaagaggaaacttcggggcccagagccagtggccacagaggcctgtgggagtggggatacaccagggggtccctgtgactccaccctgggtggggacattcgggactcagatccagagcaggccccaaccccccaggaagctgcttctgcctcagctcctccatgtgggggccccagtgctgaggcctaccttctacaccctgccgcttttcatggcgcccccagtcacctaccagccaggacccccagcttcgctgaggctccagaccctgggcgcccagccccctactcagctgcatttctggacctacagcctggaccagggggctctgcctatcaggcagctccatctgtaccatcctttgccccacacttcatccaagggggtcccttccctctaccgtacccaggacctggaggttatctggacatgggatccaagccaatgtactgagcctcggtgtagtaaccctatgccatcttcccttgatcctccagctcccttcccccagcctggtagcatccgcattgaagtggtatccccctcccccccaccaaatggctggcttgggcctctcttccaccctttagttcacaccttgatttcactccaccccctctggcttcaaagctcaggcaaggcagctctgagtccagctggggcctgcttcccctcagctctcaccttagtgcgaatggagggagcctctgcctggccaaatggggctcccagcccagtacccccacctcctgggggccttgttactagtgcaagccatgccaatttgttctcaggatcagagtatttttgttaataaaactccaaagacattaaaaaaaaaaaaaaaaa

SEQ ID NO: 4 - protein, 436 aa, Mus musculus
MYHPRELYPSLGTGYRLGHPQPGADSTFPPALTEGYRYPDLDTSKLDCFLSGIEAAPHTLAAAAPLPLLPSALGPETAPPPPEALHSLPGVSLSLENQELWKEFSAVGTEMIITKAGRRMFPACRVSVTGLDPEARYLFLLDVVPVDGARYRWQGQHWEPSGKAEPRLPDRVYIHPDSPATGAHWMRQPVSFHRVKLTNSTLDPHGHLILHSMHKYQPRIHLVRATQLCSQHWGGVASFRFPETTFISVTAYQNPRITQLKIAANPFAKGFRENGRNCKRERDARVKRKLRGPEPVATEACGSGDTPGGPCDSTLGGDIRDSDPEQAPTPQEAASASAPPCGGPSAEAYLLHPAAFHGAPSHLPARTPSFAEAPDPGRPAPYSAAFLDLQPGPGGSAYQAAPSVPSFAPHFIQGGPFPLPYPGPGGYLDMGSKPMY

SEQ ID NO: 5 - DNA, 4230 bp, Homo sapiens
gaagggtcgggggatccctccgccgccagcgcgtggtcccggccccctccacccgccgtctcggccgcggccagcagcccctgccccccgggggacgctgacggccgcccggcgcgccgccctagcagacggacagggggcgctgcgcgcggcctggggcaacccgggccacaggggcaggaaagtgagggcccaggtcggcccgggcgtgcaggggccccgggttcgcagcggcggccgcggcagcgatagcggcactagcagcagcgggagtgccgggttgagccgggaagccgatggcggcggctgcggcggctccgattcctcgctgactgcccgtccgccctcctgcatcgagcgccatgttaccgacccaagctggggccgcggcggctctgggccggggctcggccctggggggcagcctgaaccggaccccgacggggcggccgggcggcggcggcgggacacgcggggctaacgggggccgggtccccgggaatggcgcggggctcgggcccggccgcctggagcgggaggctgcggcagcggcggcaaccaccccggcgcccaccgcgggggccctctacagcggcagcgagggcgactcggagtcgggcgaggaggaggagctgggcgccgagcggcgcggcctgaagcggagcctgagcgagatggagatcggtatggtggtcggtgggcccgaggcgtcggcagcggccaccgggggctacgggccggtgagcggcgcggtgagcggggccaagccgggtaagaagacccggggccgcgtgaagatcaagatggagttcatcgacaacaagctgcggcgctacacgaccttcagcaagaggaagacgggcatcatgaagaaggcctatgagctgtccacgctgacagggacacaggtgctgttgctggtggccagtgagacaggccatgtgtatacctttgccacccgaaaactgcagcccatgatcaccagtgagaccggcaaggcactgattcagacctgcctcaactcgccagactctccaccccgttcagaccccacaacagaccagagaatgagtgccactggctttgaagagacagatctcacctaccaggtgtcggagtctgacagcagtggggagaccaaggacacactgaagccggcgttcacagtcaccaacctgccgggtacaacctccaccatccaaacagcacctagcacctctaccaccatgcaagtcagcagcggcccctcctttcccatcaccaactacctggcaccagtgtctgctagtgtcagccccagtgctgtcagcagtgccaatgggactgtgctgaagagtacaggcagcggccctgtctcctctgggggccttatgcagctgcctaccagcttcaccctcatgcctggtggggcagtggcccagcaggtcccagtgcaggccattcaagtgcaccaggccccacagcaagcgtctccctcccgtgacagcagcacagacctcacgcagacctcctccagcgggacagtgacgctgcccgccaccatcatgacgtcatccgtgcccacaactgtgggtggccacatgatgtaccctagcccgcatgcggtgatgtatgcccccacctcgggcctgggtgatggcagcctcaccgtgctgaatgccttctcccaggcaccatccaccatgcaggtgtcacacagccaggtccaggagccaggtggcgtcccccaggtgttcctgacagcatcatctgggacagtgcagatccctgtttcagcagttcagctccaccagatggctgtgatagggcagcaggccgggagcagcagcaacctcaccgagctacaggtggtgaacctggacaccgcccacagcaccaagagtgaatgatccgcccgccgccctggacagatggcccaagggatggcaccacttatttattgttgccttttcacgttttctttacacacacgttgacgggccgcaggagggaggcggggaggaggaacgggcagccacaggactgagccctctcactccagccaaagaaatgggcctgcctgcctccacccgtcctccctcagcctccccttcttcccgccccacctcccatttctgttgctggaggggctgtcctccttcctgggaccccctcgccagcttggctcgatgtttgccatgagtattagcttacccaatgggaccgtgccccacctccccacacacaggccttctgtggggctgggcaccgtgtcctcctctgaggaagcagttggggccctcttgccagcctccttgctgaccccaggtcagccctgtgtctgtcacaggctgggtcaaaagagccctggctctgcccctcagggggccagctggggagatgggggcttcttcctcacactgctgtcctctcccccttcagctcctgagtagctgggcctgtgcactgggcaggttcctggggccgcctgccctgccttgccgctccccttggacctccaggggctcctgggttggagggaaccaccagcgttcccttctcccccttgtcttcccccctctcctcccagctgctttacttaaagttgattttgaactttttatttgaggagacgaagtgaaaacaaatctataaatatatatttttaaaatatttaacttttttttatggcgtttttctcgtccccctccctgcccaaactccccttccctggggagccctcaggctccccagaactggctgggcccctggggacagagccaccccatgagctcggggtccaccagtgtgtgggggagattctgggtttgcccagtcctgggttgtttccaggagaaagccgggggaggggccctcaggccattccccaacggggtggggagggtgacccacagctctgggcctctttttgccctttagggctgttgctagggagagggaagagggagaccaaatgtcggggttggggtgggagggcgtcaggcagaggcaactgacttcatttgtgccacacgcatgggcattgcagccttgcgctgtcccaggcatgcagctgcctggggcccaagttgcagtgagcagggtggggtctgggagggggtgagaggcaggaatgggggtcagaagaagtgggagcagcttcttgggctgagtgcagccaaaggggagccagaaatgggcagttctcccagggagtgagcagctactgtaacttttttaaattaagacaaaaagccttgaagaaaatgactttatttttctaagtgtaacctcagtatttatgtaatttgtacaggggccatgccccacccccctcctccccctttggggtagaccttgagggtgggccagcataggggggagggtcttttaccctgtgtcagagcctaccttcaccacctatatccagaaggggagctttttcagaaacagggcagcagtggggtgaaattttcttaacccctaagactgccttcagtaggaacaagctggcttctgtgattaggtgaagggatgggggaagattttatgcacagcctagttatcaaggggatgatttgccgacatgtttgagaaccccctaacctctaaccctcattgctgtcttgccccagtttggggtgccaagatggaagtcacctttctgggctttctcctggagatagctggggcttatgggtggctttcaaggctggggcatggcaaatcaggggccagagagcaggggagcttgggactcaggtctgtaactgcccagccccttttctctgctcttgtttcactccaccatcactcactcactccccactcccccacccatggggaggagacctttgatgaattcttcctctccttcccacaaaagacagacccagtgagtgaatcaggcaaagtgcttataatgtgtgttgtgtgagcgtggccttgggaggacatgcgtgtgtcagggatgagttgaggtgatatttttatgtgcagcgacccttggtgtttcccttcctcggtggctctggggtatgtgtgtgtgggtgtgtgcgcctgagtgagtgtgtgtgcttgaatgtgagtgtgtatgtcagtggtttctacttcccctgggatgctgacccaggaatagtggacatggtcacagtcctatgtacagagctttcttttgtattaaaaaaaaatactctttcaataaatgtatcatttttgtgcacagaaaaaaaaaaaaaaaaaaaaaaaaaa

SEQ ID NO: 6 - protein, 508 aa, Homo sapiens
MLPTQAGAAAALGRGSALGGSLNRTPTGRPGGGGGTRGANGGRVPGNGAGLGPGRLEREAAAAAATTPAPTAGALYSGSEGDSESGEEEELGAERRGLKRSLSEMEIGMVVGGPEASAAATGGYGPVSGAVSGAKPGKKTRGRVKIKMEFIDNKLRRYTTFSKRKTGIMKKAYELSTLTGTQVLLLVASETGHVYTFATRKLQPMITSETGKALIQTCLNSPDSPPRSDPTTDQRMSATGFEETDLTYQVSESDSSGETKDTLKPAFTVTNLPGTTSTIQTAPSTSTTMQVSSGPSFPITNYLAPVSASVSPSAVSSANGTVLKSTGSGPVSSGGLMQLPTSFTLMPGGAVAQQVPVQAIQVHQAPQQASPSRDSSTDLTQTSSSGTVTLPATIMTSSVPTTVGGHMMYPSPHAVMYAPTSGLGDGSLTVLNAFSQAPSTMQVSHSQVQEPGGVPQVFLTASSGTVQIPVSAVQLHQMAVIGQQAGSSSNLTELQVVNLDTAHSTKSE

SEQ ID NO: 7 - DNA, 4093 bp, Mus musculus
agccagcgcgtggtcccggccccctccacccgcggtctcggccgcggccagcagcccctgccccgcgggggacgctgacggccgcagggcgcgcctccccggcacacggacagggggcgctgcgcgcggcctggggcaaccccggccataggggcaggaaagtgagggcccaggtcggcccgggcgtgcaggggccccgggctcgcagcggcggccgcggcagcaagagcggctttagtaacagcgcgagtgccgggtggaaccgagaagccgatggcggcagctgccccgattcctcgctgacttgcccgtcagccctcctgcactgagcgccatgttaccgagccaagctggggccgcggcggctctgggccggggctcggccctggggggcaacctgaaccggaccccgacggggcggccgggcggcggcggcgggactcgcggggcgaacgggggccgggtccccgggaacggcgcggggctcggccagagtcgtctggagcgggaggctgcagcggcagcggcgcccaccgccggggccctctacagcggcagcgagggcgactccgagtccggcgaggaggaggagctgggcgccgagcggcgcggcctcaagcggagcctgagcgagatggagctcggcgtggtggtcggtgggcctgaggcggcggcggcggccgccgggggctacgggccggtgagcggcgcggtgagcggggccaagccggggaagaagacccggggccgcgtgaagatcaagatggagttcatcgacaacaagctgcggcgctacacgaccttcagcaagaggaagacgggcatcatgaagaaggcctatgagctgtccacgctgacagggacacaggtgctgttgctggtggccagtgagacaggccatgtgtatacctttgccacccgcaaactgcagcccatgatcaccagtgagaccggcaaggcgctgattcagacctgcctcaactcgccagactctccgccccgctcagaccccaccacagaccagagaatgagtgccactggctttgaagagccagatctcacctaccaggtgtcggaatctgacagcagtggggaaaccaaggacacactgaagccagcattcacagtcaccaacctgccgggtaccacctccacaatccagacagcacccagcacctctaccaccatgcaagtcagcagcggcccctccttccccatcaccaactacctggcaccagtgtctgctagtgtcagccccagcgctgtcagcagtgccaacgggactgtgctcaagagtacaggcagcggccctgtctcctctgggggccttatgcagctgcctaccagcttcaccctcatgcctggtggggcagtggcccagcaggtccctgtgcaggccattcatgtgcaccaggccccacagcaagcgtctccctctcgtgacagcagcacagacctcacgcagacctcctccagcgggacagtgacgttgcccgccaccatcatgacgtcgtctgtacccacaactgtgggtggccacatgatgtaccctagtccccatgcagtgatgtatgcccccacctcaggcctggctgatggcagcctcaccgtgctcaatgccttctctcaggcaccatccaccatgcaggtgtcccacagccaggtccaggagccaggtggtgtccctcaggtgttcctgacagcaccgtctgggaccgtgcagatccctgtctctgcagttcagcttcaccagatggctgtgatagggcagcaagctgggagcagcagcaacctcaccgagctacaggtggtgaacctggatgccacccacagcaccaagagtgaatgatccgcccgtcaccctggacagacggcccaagggacggcaccacttatttattgttgccttttcacggtttctttacacacacactgactggccgaaggagggaggcggggagaaggagcgggcagccacaggactatgccctctcactccagccaaagaaacgggcctgcctgccctccccagctgccctccttcctgctctgcctgtcatgctgttgatggtgcagctctcctccctcctcagaccccctgccagcctggctcaatttgctatgagtattagctgacccgatgggactgtgccaccttcccactcacaggccttctgtgggactgggcacagtgttcccgtccgaggaaacatttggggccctctctccagcctctttggcgacctctggttagctttgtgtctgccatggtctgggtcaaaagagcccggcccctgcccctcagggagccagctgggagagatatgggggcttgtgccctcagccgatgccctctgcccctttcagctcctgaagcagctgggcctgtgcaccgggcaggttactggggccgcctgccctgccttgcctctgttcccgcggacctgcaggggctcctgggttggagggaaccactgttagcggtctcttcctccccttggtcttcccccttctcccagctgctttacttaagagttgattttgaactttttatttgaggagatgacgtgaaaacaaatctataaatatatatttttaaaatatttaacttttttttaatggcgtttttctcatccccctccctgcccaaactcctcttggggaagctcttgggctccctagaactgtctgggcccctggggacagccaccccatgagcttggggtccaccagtgtgtgtgtgtgggagattctgggattgctccacatcctgggttctttccaggagaaaggtgggggaggggccctcaggccaagccacagaagggggatgggaggtcccgcagttctaggcctcttttgccctctagggttgttgctaggaaaagggaagatggagaccaaatgtgggggtggggtgggaggggtcagggagaggagattgatgtcctttgtgccacagtatggtcgttgcagcctgtgctcccccaggcttgcagatacctggggcccaaggtgcagtgggcaggatggagttcgggaggtagggagatacaggaatgggggtcagaggtaaaagctcttgggctgagtgcagcaatggggggccagaggtgggcagctctcccagggagtgagcagctactgtaacttttttaaattaagacaaaaagccttgaagaaaatgactttatttttctaagtgtaacctcagtatttatgtaatttgtacagggccatgccccatcccttaaaatccctttggggaccttgagggtgggctagcataggggagggtgttttaccctgtgtcagagccagcctaaaccacctgtatccattaaaggagctttttccgggagggcagttggggtagggtgtcactaaacccttgttgctgccttcagtaggaacaagcctacttctgcggttaggtggaggggttgggaaggttttatgcacagcctacgtttcatggggatgatctgctgacgtttaagagccccctgaacctctaatccccattactgtcttccccagctgggatgccacctttctggcctttctcctggaaatagctggggcgtttgggtggctttcaagactggggcatggtaaaccaggggtcagagttggggagcttgggactcaggtctgtaactgcccagcccccttctctgctcctgtttcactcctctgccgcccactcatggcccctcccccaacaccggaggtgttgattcctcctcacaaaagatagagcctgggcaagtgattcaggccaagtgcttaccaacgtgtgctgtgtgattgtggccttgggagaacctgtttgtcaggcaggactgaggtggcatttctatgtgcagcaacccttggtgtatccctaattaagtggctctaggggtgtgtgcgcgcgcctgtgtccgagtgaatacgtgtgtgaatgtggagtgcgtgtggtatatccgtggtttctacttctcctgggatgttgacccaggaatagtggacatggtcacatcttctgtacagagctttcttttgtattaaaaaaatactctttcaataaatgtatcatttttgtgcacag

SEQ ID NO: 8 - protein, 504 aa, Mus musculus
MLPSQAGAAAALGRGSALGGNLNRTPTGRPGGGGGTRGANGGRVPGNGAGLGQSRLEREAAAAAAPTAGALYSGSEGDSESGEEEELGAERRGLKRSLSEMELGVVVGGPEAAAAAAGGYGPVSGAVSGAKPGKKTRGRVKIKMEFIDNKLRRYTTFSKRKTGIMKKAYELSTLTGTQVLLLVASETGHVYTFATRKLQPMITSETGKALIQTCLNSPDSPPRSDPTTDQRMSATGFEEPDLTYQVSESDSSGETKDTLKPAFTVTNLPGTTSTIQTAPSTSTTMQVSSGPSFPITNYLAPVSASVSPSAVSSANGTVLKSTGSGPVSSGGLMQLPTSFTLMPGGAVAQQVPVQAIHVHQAPQQASPSRDSSTDLTQTSSSGTVTLPATIMTSSVPTTVGGHMMYPSPHAVMYAPTSGLADGSLTVLNAFSQAPSTMQVSHSQVQEPGGVPQVFLTAPSGTVQIPVSAVQLHQMAVIGQQAGSSSNLTELQVVNLDATHSTKSE

SEQ ID NO: 9 - DNA, 8580 bp, Homo sapiens
aatcgccggcagcctatgacatcagacaggaacgcctgggatgccgcgctgctcctggccaacctccgaggaggaggagggtcccgccggctaagagttaattagccccgcacggcgaggggggaggcgccagttttctggggacactggctgccactgtactcctacccaggggagctcacggagagttggatgaattctgggttgttagctgcggtcagctgggctcccgggagcctgttgctggtggagaacagggggcgcctggccaagggaccagcggcttgctgagactcaacatgacactcctggggtctgagcattccttgctgattaggagcaagttcagatcagttttacagttaagacttcaacaaagaaggacccaggaacaactggctaaccaaggcataataccaccactgaaacgtccagctgaattccatgagcaaagaaaacatttggatagtgacaaggctaaaaattccctgaagcgcaaagccagaaacaggtgcaacagtgccgacttggttaatatgcacatactccaagcttccactgcagagaggtccattccaactgctcagatgaagctgaaaagagcccgactcgccgatgatctcaatgaaaaaattgctctacgaccagggccactggagctggtggaaaaaaacattcttcctgtggattctgctgtgaaagaggccataaaaggtaaccaggtgagtttctccaaatccacggatgcttttgcctttgaagaggacagcagcagcgatgggctttctccggatcagactcgaagtgaagacccccaaaactcagcgggatccccgccagacgctaaagcctcagataccccttcgacaggttctctggggacaaaccaggatcttgcttctggctcagaaaatgacagaaatgactcagcctcacagcccagccaccagtcagatgcggggaagcaggggcttggcccccccagcacccccatagccgtgcatgctgctgtaaagtccaaatccttgggtgacagtaagaaccgccacaaaaagcccaaggaccccaagccaaaggtgaagaagcttaaatatcaccagtacattcccccagaccagaaggcagagaagtcccctccacctatggactcagcctacgctcggctgctccagcaacagcagctgttcctgcagctccaaatcctcagccagcagcagcagcagcagcaacaccgattcagctacctagggatgcaccaagctcagcttaaggaaccaaatgaacagatggtcagaaatccaaactcttcttcaacgccactgagcaatacccccttgtctcctgtcaaaaacagtttttctggacaaactggtgtctcttctttcaaaccaggcccactcccacctaacctggatgatctgaaggtctctgaattaagacaacagcttcgaattcggggcttgcctgtgtcaggcaccaaaacggctctcatggaccggcttcgacccttccaggactgctctggcaacccagtgccgaactttggggatataacgactgtcacttttcctgtcacacccaacacgctgcccaattaccagtcttcctcttctaccagtgccctgtccaacggcttctaccactttggcagcaccagctccagccccccgatctccccagcctcctctgacctgtcagtcgctgggtccctgccggacaccttcaatgatgcctccccctccttcggcctgcacccgtccccagtccacgtgtgcacggaggaaagtctcatgagcagcctgaatgggggctctgttccttctgagctggatgggctggactccgagaaggacaagatgctggtggagaagcagaaggtgatcaatgaactcacctggaaactccagcaagagcagaggcaggtggaggagctgaggatgcagcttcagaagcagaaaaggaataactgttcagagaagaagccgctgcctttcctggctgcctccatcaagcaggaagaggctgtctccagctgtccttttgcatcccaagtacctgtgaaaagacaaagcagcagctcagagtgtcacccaccggcttgtgaagctgctcaactccagcctcttggaaatgctcattgtgtggagtcctcagatcaaaccaatgtactttcttccacatttctcagcccccagtgttcccctcagcattcaccgctgggggctgtgaaaagcccacagcacatcagtttgcccccatcacccaacaaccctcactttctgccctcatcctccggggcccagggagaagggcacagggtctcctcgcccatcagcagccaggtgtgcactgcacagaactcaggagcacacgatggccatcctccaagcttctctccccattcttccagcctccacccgcccttctctggagcccaagcagacagcagtcatggtgccgggggaaacccttgtcccaaaagcccatgtgtacagcaaaagatggctggtttacactcttctgataaggtggggccaaagttttcaattccatccccaactttttctaagtcaagttcagcaatttcagaggtaacacagcctccatcctatgaagatgccgtaaagcagcaaatgacccggagtcagcagatggatgaactcctggacgtgcttattgaaagcggagaaatgccagcagacgctagagaggatcactcatgtcttcaaaaagtcccaaagatacccagatcttcccgaagtccaactgctgtcctcaccaagccctcggcttcctttgaacaagcctcttcaggcagccagatcccctttgatccctatgccaccgacagtgatgagcatcttgaagtcttattaaattcccagagccccctaggaaagatgagtgatgtcacccttctaaaaattgggagcgaagagcctcactttgatgggataatggatggattctctgggaaggctgcagaagacctcttcaatgcacatgagatcttgccaggccccctctctccaatgcagacacagttttcaccctcttctgtggacagcaatgggctgcagttaagcttcactgaatctccctgggaaaccatggagtggctggacctcactccgccaaattccacaccaggctttagcgccctcaccaccagcagccccagcatcttcaacatcgatttcctggatgtcactgatctcaatttgaattcttccatggaccttcacttgcagcagtggtagaatgcccaatgcaccagtgctatggaagaccaatggagttccatgggggaaagcacacagccatacatactttactgtccaaaaacagaagaagaagaagagaattaaaaagaagcaatgatttctgtgccaatgaacaagaacaaaagtcatttttagaaatacatatactgtaatatttaccaacagtcagtaactgttaatgatttcaacaatgcattaaaagaatgtgctttctcagattaaggatgccaaaaaagatatttcactgccttttcaaagaccagtatattttctagcccataatttttctcaggcattgttggggcataagctcacactgtaagcttttctcatgaattcactagacataacgtggaaggaaaacgtagtcttttgggagtacagggaagccagcccctcaaagcttatggaagacatacctgcaatggaagctgttgcccaatgtctccattactatctttcaaaagagaagccagacccagcttcagatcaaaagttcttgagacagaggaacaaaaccaatcgatttccagggaagctaatcaactctcttttccctctaccacaaaactgccctgctggagtggttctgaacctgtacccaggactcgatgtggtcactaataacaattaacctgaactgagtccacagaactccactcggaactttcttcttttttaactagtggcccaatcattcccaccatctctgtgctgataagtacgtgtcctagatgagaaccctgaagaatgcagaccttcttcccccgaaggagatgccacaagctctccaacacagccccctttagttccaaagactagagatgaccacattggtagaagtatatctcgaggcacaggaagggagccccaccagggataattcagacaggactagagaataacatcatttcacataccctgggataaacaccctgggttcctatagaaggactattacttatgggagtccaacttctccttttgttttgttattatcagtttatctttctcccactccacttttccttcaaggtaccaatcctttcctgttcctcgtttggccatctttctttttctgcctccacattgggaggggaggacttctcagttctaacaagctgccatactcctaagaaagccatttttgaaaaatttaacaatccaggttcttctggagaactcattctccacacgcacagtttgctgcaaaaggaagttgcaagaatttcttgaggaagaaactggtgacttggtccatcagtcacgaagttctttctattctcgtttagttttcaagaaattattggtttgtgttgctctggggaaattggaaatcattacattgtaaagacaaatatggatgatatttacaagagagaatttcagatctgggtttttgaaagaaaacagaattgcgcattgaaaacgatggaaggaaaaagacaatggtctaatgtgcattcctcattacctctcgtggctttggctgggagttggaaaaagctaaaatttcagaacagtctctgtaaggctctctgtggctccagttcaccattttatattgttgcatgctgtagaaaggagctattgctgttgttttgtttttttatttaaatcactaaggcactgtttttatcttttgtaaaaaaaaaaaaaaagttgttcactgtgcacttatagaaaaaataatcaaaaatgttgggattttagaagctctctttttgataaaccaaagatttagaagtcattccattgttaacttgtaaaaatgtgtgaacacagagagtttttggtgattgctactctgaaagctgccagatcttattctgggggtgggatgtggaggaatacacatacacacacaaacatacatgtatgtataatagatatatacatatgtgtatattatatctgtgtgtgcatgtatctccaaaagcggcgttacagagttctacaccaaaagcctttaacccttaatctgctgtgaatgatacctggcctttctcactatgaatttctgattaaccaaccagactacacgttgcctctctgtgtatgactaacggctccaacccgatgactcacagctacttgcttatcgtgaacaagctcatcttggcaatgaatatggatgtgaaaagacagaacagcttcaccattagtagctggaaatggtatcacagtctcttatagaggaatatgaaaggaacaagaaaatcattttacattccttttatctgtattgtgctttaaaagatccacatggtaaattttttattttgcttttatgtcagtcatcagaaccaaaaaaatccagaagaaaaaattgccagtgtttcctttgaagatgaagctactggggaagaaaaccttattaatacactccacacatttgttcattcctcagctgttggtgttttcttggggtcttgacaaagcttgctggtcagtgcacttttcaggtgtcacgttttgctgtttgtatgttttttcttccccttacttcctttggaaaacaaactcacacagtgcccctactctgagacctgggactgagtgttaattattttttccttgggtatttctatctgagagactagacctagttaggaggcctctgtacttctccagattgtacctttttatggggatctttgaggctatgacccaggactgatagatatgccttacggaagacaaaagataaaatggttcctatatcctaatgcaaaccaacacagttaaaagagcagatctctggataactgctctcaacctgcttctacagtctccacaaaccgcattcaccctctctcttcatagctcagacatgaaatttgagggagaaaactggagataattgggagaaaattgatgaagttggctgcttccagtagatcagataatccatgaatttgtctcccattgagaattttattttaaattcttttaaactcttcgttgtgtcttttgtgatgacaaatcaggcatgactaaaagatgtacagagacttacgaagatggtcacattcaagttccctaatgctcttagaacctgaagatgaccatgtgtagttttcttaagacctctgaacccccatggtgatgaagacttgaagacatttgcagctatctgctgcagtctggtagattcatacttatctaaagaagtcaaaaaatttattcgtgcaagtgcttgcaggaagccagtgcttattagtagtgaccctgcttctatcaacgttattgagacaacacatattctattctaagggagaaagagggaggaagagagggagggagggaggaagaagagggagggagcgaggaaggaagataggagatgggtaggggggtaaagagaaaaggagggagaagggaaggaaggaaagaagagaggaaagaaaggagggaaggaaaaaagggccaaactttctgatctatgaacttctcagttcagctgtcacattatgagaagtaaatcagaatttttttaaggagaagtcattcttagcactacaataattgtaccagtaattgaggaaaccaagacaatcttcacctgaataatagagggtctgagaactgtcagccttttgccattcaaaaacatttatgtccaacctgaaaaaaaagcatcaataaaacctatcccaagcattcaaaatagtcctttccaaatgttatttattttaaagtcaatcagctcttttagaaacagattctggtctggctgaaaactcccacaacaaatttactcatccagtggctaatatttaatgcccaccatgggcagagcacacaaatcttcagataaacaatactgaattgattcagcacagattgttcagatttgaatgaccagggagttgtatttgcacatgcaagaacactaagaatctcccagtcctcaaactagaaacctttcagctacgatgaaaaaaaaaaaggggtcttcatttttccaagagggggtggaggtggggatcactttttagctaaaagctatctctcacttcaaaattcttgtctttttctttgtggacaaacaccagtagtctatcacttggagatcttttaatatctcccatcatttaaaacatccacgagagtttgaagatttgtgttgattgccagatacagaagccccttgaaaataaggaaagggtggaggaagcatttttgtgtcctatccctacttatcgtagcagctctatagacaaaagggacacttactggtgagcctctggcccttaaaagaaaatcatctaagaatatgaaggcaatttgatttccccccacagccctcagctgccttcctcacagaaggaagttcccaaaattgctggtacacagtttgcaatcaaatatcagatatgagaaaacctgtagtgaagagtctgggttcttggttttctcataaatccaatataaatttgtaggttggttcagggtcaaaattgccagtgctttattagacagatgatactgatagacacacagagcccaggtcctggaacaagacaatcctgtagtgccaagatctggtcagttgcgttaaggagctgggtttgattctagagtccaggtttatagagaaaccctggctagattgagcctacccatggggagacgatttcaagacaggatgagatctgggaagaattttgttgtcatctgccagggaaattatcacaggactcattgaatgcaataacatgtgagtaagttcccttttgattctgggaatcagcgattttccctgtggattaagacaaaccaacgccagaaggtctcctgtgcttattttaaccatctgctcccatcgtgaaccctggagcatgcatttcctagaagtggtttcatagctcctgtgtgttcatggaaaaggggagtataatgatggggatgctggaagcttttttaatgttttccaaaggaaaggaacccacactgctccccagagttcctttccaatggccctgcagtaagaacggaggacaatgtattgctgggtgcttaaaatcctccctcagtgaagcacaaagagacactttgtaaagaaaaaaagagcaagcataggttctctgtgggaccttgtggagtggtgttttcacgttggtctctttggctcaattgagcataatcagaaagaaatgtgggttattgggaagagacaaaaagcagtggctaaaataccaaagttggcatgtgttcttttttaaaaaaaaaaaaaaatgcatatatttttaaataaaatgtttattttaaaaagaaaaaaaaaaaaaaaaaa

SEQ ID NO: 10 - protein, 986 aa, Homo sapiens
MTLLGSEHSLLIRSKFRSVLQLRLQQRRTQEQLANQGIIPPLKRPAEFHEQRKHLDSDKAKNSLKRKARNRCNSADLVNMHILQASTAERSIPTAQMKLKRARLADDLNEKIALRPGPLELVEKNILPVDSAVKEAIKGNQVSFSKSTDAFAFEEDSSSDGLSPDQTRSEDPQNSAGSPPDAKASDTPSTGSLGTNQDLASGSENDRNDSASQPSHQSDAGKQGLGPPSTPIAVHAAVKSKSLGDSKNRHKKPKDPKPKVKKLKYHQYIPPDQKAEKSPPPMDSAYARLLQQQQLFLQLQILSQQQQQQQHRFSYLGMHQAQLKEPNEQMVRNPNSSSTPLSNTPLSPVKNSFSGQTGVSSFKPGPLPPNLDDLKVSELRQQLRIRGLPVSGTKTALMDRLRPFQDCSGNPVPNFGDITTVTFPVTPNTLPNYQSSSSTSALSNGFYHFGSTSSSPPISPASSDLSVAGSLPDTFNDASPSFGLHPSPVHVCTEESLMSSLNGGSVPSELDGLDSEKDKMLVEKQKVINELTWKLQQEQRQVEELRMQLQKQKRNNCSEKKPLPFLAASIKQEEAVSSCPFASQVPVKRQSSSSECHPPACEAAQLQPLGNAHCVESSDQTNVLSSTFLSPQCSPQHSPLGAVKSPQHISLPPSPNNPHFLPSSSGAQGEGHRVSSPISSQVCTAQNSGAHDGHPPSFSPHSSSLHPPFSGAQADSSHGAGGNPCPKSPCVQQKMAGLHSSDKVGPKFSIPSPTFSKSSSAISEVTQPPSYEDAVKQQMTRSQQMDELLDVLIESGEMPADAREDHSCLQKVPKIPRSSRSPTAVLTKPSASFEQASSGSQIPFDPYATDSDEHLEVLLNSQSPLGKMSDVTLLKIGSEEPHFDGIMDGFSGKAAEDLFNAHEILPGPLSPMQTQFSPSSVDSNGLQLSFTESPWETMEWLDLTPPNSTPGFSALTTSSPSIFNIDFLDVTDLNLNSSMDLHLQQW

SEQ ID NO: 11 - DNA, 5127 bp, Mus musculus
tggcagcctatgacatcagccaggaacgcctaggatgcggctgttcccggccaccctcagaggaggagggtcctgcctgctgggagttaattagcctcgcgagcggcgaggggggaggcgccagttttctggggacactggcggccactgtgcgtcctcctacccaagggagctccccaagagttggatgaattctgggttgttagctgctgtcctctgggctcccgggagccagtttctggtggaaagcggggcgcctggccaacgaccagcggcttgctgagactcaccatgacactcctggggtctgaacactctttgctgattagaaggaagttccgatcagtcttacagttacggcttcaacagagaaggacccaggagcagctggctaaccaaggcttaataccgccactgaaaggtccaactgaattccatgacccgagaaaacaattggatagtgccaagactgaagattccctgaggcgcaagggcagaaacaggtccgaccgtgccagcctggttactatgcacattctccaagcctccacggcagaaaggtccattccaactgctcagatgaagctcaaaagagcccgccttgcagatgacctcaatgagaagatcgctctccgcccagggcccttggaactggtggagaagaacattctgccgatggattcttccgtgaaagaggctataaaaggtactgaggtgagcctctccaaggcagcagatgcattcgcctttgaggatgacagcagtagagatgggctctctccagatcaggctaggagcgaggacccccagggctctacaggatccaccccagacatcaaatccactgaggctcctctggacacaatccaggatctcactcctggctcagaaagtgacaagaatgatgcagcctcccagccaggcaaccagtcagaccctgggaagcaggttctcggccccctcagcaccccgattcctgtgcacactgctgtaaagtccaagtctttgggtgacagtaagaaccgccacaaaaagcccaaagaccccaaaccaaaggtgaagaagctcaaataccatcagtacatccccccagaccagaaggcagagaagtctcccccacccatggactctgcctatgcccggctgctccagcaacagcagctattcctgcagctacagatcctcagccagcagcagcaacagcagcagcaacagcagcagcagcaacagcagcagcagcagcagcagcagcggttcagctaccctgggatgcaccaaacacacctcaaagaaccaaatgaacagatggccagaaatccgaatccttcttcaacaccactgagcaatacccctctatcccctgtcaaaaatagcatttctggacaaactggtgtttcttctctcaaaccaggccccctcccacccaacctggatgatctcaaggtgtcagagttaagacaacagcttcgaatccggggcttgccagtgtcaggcaccaagacagcgctggtggaccggcttcgtcccttccaggattgtgctggcaaccctgtgcccaactttggggacatcacaactgtcacctttcctgtcacgcccaacaccttgcccagttatcagtcctccccgacaggcttctaccactttggcagcacaagctccagcccacccatctcccccgcctcatctgacttgtccgctgcagggtccctgccagacaccttcaccgatgcgtcacctggcttcggcctgcacgcatctccggtgcccgcctgcacggacgagagtctgctgagcagcctgaatgggggctcgggcccctccgagcctgatgggctagactctgagaaggacaagatgctggtggagaagcagaaagtgatcaaccagctcacctggaagctgcggcaagagcagcggcaggtggaagagctgagaatgcaactgcagaagcagaagagcagctgcagcgaccagaagccactgcccttcttggccaccaccatcaaacaggaagatgtctccagctgccccttcgcaccccagcaggcgtctgggaagggacagggccacagctctgacagtccccctccggcttgtgagacggctcagctgctgcctcactgtgtggagtcctcaggtcaaacccatgtactctcgtccacgtttctcagcccccagtgctcccctcagcactcgcccctggggggcctgaagagcccgcagcacatcagcctgcctccatcacccaacaaccattacttcctggcttcctcttcgggagctcagagagagaaccatggggtctcttcacccagcagcagccaagggtgcgcacagaactcaggggcacacgaaggccattcttctagcttctcttccccagcttccagcctccatcagcctttctctggcacccaagcagacagcagtcacagtgctgggctcaacccttgtcccaaaagcccaagtattcatccaaagatgactggtttacaatcttctgacaaggtggggccaacgttttcaattccatccccaactttttctaagtcaagttcagcagtttcagatatcacccagcccccatcctatgaagatgcagtgaagcagcaaatgactcggagtcagcagatggacgaactcctggatgtcctcattgaaagtggagaaatgccagccgatgccagggaagatcattcatgtcttcagaaaattccaaagatccctgggtcctcctgcagcccaactgccatccccccgaagccctcggcttcctttgagcaggcatcttcgggaggccagatggccttcgatcactacgccaacgacagtgacgaacacctggaagtcttattgaattctcacagccccatcggaaaggtgagcgatgttaccctcctcaaaatcggaagcgaggagcctccttttgacagcatcatggatggcttcccagggaaggctgcggaagatctcttcagtgctcacgagctcttgcctgggcccctctccccgatgcatgcacagttgtcacctccttctgtggacagcagtggtctgcagctgagcttcacggaatctccttgggaaacaatggaatggctggacctcactccacctagttccacgccaggcttcagcaaccttacctccagtgggcccagcattttcaacatcgattttctggatgttacagatcttaatctgaattcccctatggatctccacttacagcagtggtaaacacccgaggtacaagagctacgagagctcagtgggaattcaatggaggaaagcacgataccggaaatgtgtgttccaaaagatgaagtggggaaaatggggagggaaaaaaaaaaacagcaacggaggtttttgtgacaactaaccagaacaaacagaagtcagctattaaaatatgtctaaatgtaatatctaccagcattcagtaactgttaataacttcagtgatgcattcaaaaatgtgctttgtcagaataagaatgccaaaaatgttttttcgctgccttatctcataccagtttttttggggttttttttgtttgtttgttttttggttttttttttttttgtgttgttatttggttttctttttgcccacagtttgtctcaggcaatactgggacatagactgaccccattagcttttgttatgaatttactaaactttctgtggaaggagaacagagcctctgccgcgggtgtggggaagccatcctgtgcttgaggcagcacacgtgtgtccatcatcatcagtcagaagagcagggcctgtctcacccaatcgagtccttaagacagaataatcagaatggtcagagggacagaccaatcaattcccaggaaagcaaaagtgactcaatgtcccttgactcccaaatggtcccactggactggtgatcactggtgacaactaactagctttgtccagagaatccacccagaacacggtgctttttagccagtagtccacctctatgtgcatcagcaatgcatagcaggtgagaacttgaatcacagaaacttcatgccatggatggagactcctgaggcgctcaaatactactacctctagttccaaagactagagctagatgatcagaaaggcaactggaggcccagggagccgtactgggacaagttagaattagagaacgatgtcatttaacattccgagaaagaaataaccatgaattgctattacaggagtaacacacagggccagcttctttttcttcttttttatttttcttttcttattgtgagcagagggaattcacctcagttcatctttctctcagtacttttctttcaagatatcaatcctttatgactcttttgcttttaattctctctctctctctctctctctctctttctctcaaaggagaggtttcagttctaacaagctaccatactcctattaaagccatttttttttttagaatattaaaagtccaaactctcttgccaaactctttcttcacatgcgcattggctgaaaacagaatttacaagaatttctttaggaagaaactggggatgtggcccattggtcacaaagtttttttgtttgtttttgtttttgtttcaattcttgtttgatttatggacaatctttggtttgtattgctctggagaaattggaaatcattgcagagtgaagataaatcagggcaccatgtatagtagagaatatttcagtagttttccaaacgagaacacaattgcacactgtaaacaacaggagtgtgaaggaccacagtcttgaggagttcttgttgccctgcgtttggtgaaggcgttggggaccgaggaagacaacatacagtttggccaaggctctcagaggcttgctgtggcgccaattcaagtattacaatgttgcatgctgtagaaagtagctgttgctgttgttttgttttgttttaatttaagtcaccaaggcactgttttattcttttgtaaaaaaaaaaaaaagttcactgtgcacttatagagaaaataatcaacaatgttgtgaatttttgagaagactttttttttttttttgataaaccaaagatttagaaatcattccattgtcaacttgtaaaaaaaaaaaaa

SEQ ID NO: 12 - protein, 983 aa, Mus musculus
MTLLGSEHSLLIRRKFRSVLQLRLQQRRTQEQLANQGLIPPLKGPTEFHDPRKQLDSAKTEDSLRRKGRNRSDRASLVTMHILQASTAERSIPTAQMKLKRARLADDLNEKIALRPGPLELVEKNILPMDSSVKEAIKGTEVSLSKAADAFAFEDDSSRDGLSPDQARSEDPQGSTGSTPDIKSTEAPLDTIQDLTPGSESDKNDAASQPGNQSDPGKQVLGPLSTPIPVHTAVKSKSLGDSKNRHKKPKDPKPKVKKLKYHQYIPPDQKAEKSPPPMDSAYARLLQQQQLFLQLQILSQQQQQQQQQQQQQQQQQQQQQRFSYPGMHQTHLKEPNEQMARNPNPSSTPLSNTPLSPVKNSISGQTGVSSLKPGPLPPNLDDLKVSELRQQLRIRGLPVSGTKTALVDRLRPFQDCAGNPVPNFGDITTVTFPVTPNTLPSYQSSPTGFYHFGSTSSSPPISPASSDLSAAGSLPDTFTDASPGFGLHASPVPACTDESLLSSLNGGSGPSEPDGLDSEKDKMLVEKQKVINQLTWKLRQEQRQVEELRMQLQKQKSSCSDQKPLPFLATTIKQEDVSSCPFAPQQASGKGQGHSSDSPPPACETAQLLPHCVESSGQTHVLSSTFLSPQCSPQHSPLGGLKSPQHISLPPSPNNHYFLASSSGAQRENHGVSSPSSSQGCAQNSGAHEGHSSSFSSPASSLHQPFSGTQADSSHSAGLNPCPKSPSIHPKMTGLQSSDKVGPTFSIPSPTFSKSSSAVSDITQPPSYEDAVKQQMTRSQQMDELLDVLIESGEMPADAREDHSCLQKIPKIPGSSCSPTAIPPKPSASFEQASSGGQMAFDHYANDSDEHLEVLLNSHSPIGKVSDVTLLKIGSEEPPFDSIMDGFPGKAAEDLFSAHELLPGPLSPMHAQLSPPSVDSSGLQLSFTESPWETMEWLDLTPPSSTPGFSNLTSSGPSIFNIDFLDVTDLNLNSPMDLHLQQW

(19) United States
(12) Patent Application Publication
(10) Pub. No.: US 2019/0071641 A1
(43) Pub. Date: Mar. 7, 2019

(54) METHOD FOR DIRECTLY PRODUCING CARDIAC PRECURSOR CELL OR MYOCARDIAL CELL FROM FIBROBLAST

(71) Applicants: Keio University, Minato-ku, Tokyo (JP); National Institute of Advanced Industrial Science and Technology, Chiyoda-ku, Tokyo (JP)

(72) Inventors: Masaki IEDA, Tokyo (JP); Taketaro SADAHIRO, Tokyo (JP); Mari ISOMI, Tokyo (JP); Naoki GOSHIMA, Tokyo (JP)

(73) Assignees: Keio University, Minato-ku, Tokyo (JP); National Institute of Advanced Industrial Science and Technology, Chiyoda-ku, Tokyo (JP)

(21) Appl. No.: 16/084,427

(22) PCT Filed: Mar. 7, 2017
(86) PCT No.: PCT/JP2017/009052

§ 371 Date: Sep. 12, 2018

(30) Foreign Application Priority Data
Mar. 15, 2016 (JP) 2016-051407

(51) Int. Cl. C12N 5/077; C12N 5/10; C12N 15/63; C12N 15/52; C12N 5/071

(52) U.S. Cl. CPC C12N 5/0657; C12N 5/10; C12N 15/63; C12N 2506/1307; C12N 5/0661; C12N 15/52; C12N 5/069; C12N 5/0656

(57) ABSTRACT

Provided is a method of inducing cardiac progenitor cells or cardiomyocytes from fibroblasts. The present invention provides a method for producing cardiac progenitor cells, comprising introducing one cardiac reprogramming factor into fibroblasts, or a method for producing cardiomyocytes, comprising introducing three cardiac reprogramming factors into fibroblasts.

### TECHNICAL FIELD

The present invention relates to a method for producing cardiac progenitor cells and cardiomyocytes from fibroblasts; and fibroblast-derived cardiac progenitor cells and fibroblast-derived cardiomyocytes, which are produced by the aforementioned method.

### BACKGROUND ART

Heart disease has steadily increased with aging, and the incidence of heart failure in men aged 80 or over is high (14.7%). The heart is composed of cells such as cardiomyocytes and fibroblasts. Since cardiomyocytes having a beating function have almost no or completely no regeneration ability, the method for treating heart disease has been restricted so far.

To date, a method for directly producing cardiomyocyte-like cells from fibroblasts, without going through iPS cells, wherein the method comprises introduction of three cardiac reprogramming factors (Gata4, Mef2c and Tbx5; hereinafter referred to as “GMT”), has been found (Non Patent Literature 1). It was elucidated that, according to this method, the cardiac muscle can be directly produced from fibroblasts by the three factors GMT even in cultured cells and in the living body of a mouse (Patent Literature 1). Moreover, it has been reported that functionally immature cardiomyocyte-like cells can be produced from fibroblasts through cardiac progenitor cells, by using transcriptional factors (Mesp1 and Ets2) and a plurality of humoral factors (Non Patent Literature 2). Furthermore, a method of inducing cardiac progenitor cells or cardiomyocytes from pluripotent stem cells such as ES cells or iPS cells, using a humoral factor (Non Patent Literature 3), and a method of inducing cardiac progenitor cells from mouse ES cells, using a transcriptional factor, under the use of serum or under special culture conditions, have been reported (Non Patent Literature 4).

On the other hand, as described above, cardiomyocytes do not have proliferation ability. Accordingly, when cardiomyocytes are directly induced from fibroblasts or pluripotent stem cells, it is likely that the number of cells necessary for regenerative therapy could not be sufficiently obtained. Hence, a method for producing cardiomyocytes, comprising first producing cardiac progenitor cells having proliferation ability, and then producing cardiomyocytes from the cardiac progenitor cells, is useful.

However, a method for producing cardiac progenitor cells from fibroblasts, by using the aforementioned transcriptional factors (Mesp1 and Ets2) and a plurality of humoral factors, has been problematic in that the state of cardiac progenitor cells cannot be maintained, and that only functionally immature cardiomyocyte-like cells can be produced by the method.

### CITATION LIST

### Patent Literature

Patent Literature 1: WO2011/139688

### Non Patent Literature

Non Patent Literature 1: Ieda, M., Fu, J. D., Delgado-Olguin, P., Vedantham, V., Hayashi, Y., Bruneau, B. G., and Srivastava, D. Direct Reprogramming of Fibroblasts into Functional Cardiomyocytes by Defined Factors. Cell 142: 375-386. 2010.
Non Patent Literature 2: Islas J F, Liu Y, Weng K C, et al. Transcription factors ETS2 and MESP1 transdifferentiate human dermal fibroblasts into cardiac progenitors. Proceedings of the National Academy of Sciences of the United States of America 2012; 109(32): 13016-21.
Non Patent Literature 3: Kattman S J, Witty A D, Gagliardi M, et al. Stage-specific optimization of activin/nodal and BMP signaling promotes cardiac differentiation of mouse and human pluripotent stem cell lines. Cell stem cell 2011; 8(2): 228-40.
Non Patent Literature 4: van den Ameele J, Tiberi L, Bondue A, et al. Eomesodermin induces Mesp1 expression and cardiac differentiation from embryonic stem cells in the absence of Activin. EMBO reports 2012; 13(4): 355-62.

### SUMMARY OF INVENTION

### Technical Problem

Under the aforementioned circumstances, it has been desired to develop a method of inducing cardiac progenitor cells from fibroblasts, in which the induced cardiac progenitor cells are maintained for a certain period of time, and a method for producing functionally mature cardiomyocytes from the induced cardiac progenitor cells.

### Solution to Problem

As a result of intensive studies directed towards achieving the aforementioned objects, the present inventors have found that, when one factor (Tbx6) is introduced into fibroblasts, using a retroviral vector and retrovirus, cardiac progenitor cells, which express Mesp1 as a marker for the cardiac progenitor cells, are induced, and then, the induced cardiac progenitor cells proliferate to form a colony. In addition, the present inventors have also found that, in the induced cardiac progenitor cells, the expression of a plurality of cardiac progenitor cell gene markers is maintained even 1 month after the induction.

Moreover, the present inventors have found that, when three factors (Tbx6, SRF, and Myocd) are introduced into fibroblasts using a retroviral vector and retrovirus, beating cardiomyocytes are induced, and that the obtained cells express Nxk2.5 or troponin, which are markers for cardiomyocytes.

Furthermore, the present inventors have found that, when such three factors (Tbx6, SRF, and Myocd) are introduced into fibroblasts using a retroviral vector and retrovirus, the obtained cells express Myh11 as a marker for smooth muscle cells, and also that Pecam1 as a marker for vascular endothelial cells is expressed therein.

The present invention has been completed based on these findings.

Specifically, the present invention is as follows.

[1] A method for producing cardiac progenitor cells, comprising a step of introducing a Tbx6 gene into fibroblasts.
[2] A method for producing cardiomyocytes, comprising a step of introducing a Tbx6 gene, an SRF gene and a Myocardin gene into fibroblasts.

The above-described fibroblasts are, for example, mouse cells or human cells.

[3] A cardiac progenitor cell derived from a fibroblast, comprising an exogenous Tbx6 gene.
[4] A cardiomyocyte derived from a fibroblast, comprising an exogenous Tbx6 gene, an exogenous SRF gene and an exogenous Myocardin gene.
[5] An inducer for inducing cardiac progenitor cells from fibroblasts, wherein the inducer comprises a Tbx6 gene.
[6] An inducer for inducing cardiomyocytes from fibroblasts, wherein the inducer comprises a Tbx6 gene, an SRF gene and a Myocd gene.
[7] An inducer for inducing smooth muscle cells from fibroblasts, wherein the inducer comprises a Tbx6 gene, an SRF gene and a Myocd gene.
[8] An inducer for inducing vascular endothelial cells from fibroblasts, wherein the inducer comprises a Tbx6 gene, an SRF gene and a Myocd gene.

### Advantageous Effects of Invention

According to the present invention, provided are a method for directly producing cardiac progenitor cells from fibroblasts, and a method for directly producing cardiomyocytes from fibroblasts. In addition, it is possible to provide cardiac progenitor cells and cardiomyocytes, which are produced by the method of the present invention. Since the cardiac progenitor cells induced from fibroblasts according to the present invention have maintained the expression of a plurality of cardiac progenitor cell genes, the present invention can provide a method for producing cardiac progenitor cells, which is more stable than conventional methods, and cardiac progenitor cells. Since cardiac progenitor cells have proliferation ability, the cardiac progenitor cells produced by the present invention can be preferably applied to medical use.

Moreover, the cardiomyocytes induced by the present invention have been confirmed to beat, and further, the expression of a cardiac muscle-specific gene or the expression of a structural protein has been confirmed in the present cardiomyocytes. Therefore, the method for producing cardiac muscle of the present invention can provide functionally mature cardiomyocytes.

### BRIEF DESCRIPTION OF DRAWINGS

FIG. 1 is a schematic view showing introduction of a Tbx6 gene into fibroblasts to induce cardiac progenitor cells.

FIG. 2 shows the immunostaining (MespCre-GFP) of Tbx6 gene-introduced cells with GFP (Mesp1 expression), and with DAPI (nuclear counterstaining), and the merged image of them (Merged).

FIG. 3 is a view showing the mRNA expression levels of cardiac differentiation markers, Mesp1, T, KDR, Nkx2.5 and TnnT2, in cells induced by a Tbx6 gene, 1 month after the induction.

FIG. 4 is a schematic view showing introduction of Tbx6, SRF, and Myocd (TSM) genes into fibroblasts, to induce beating cardiomyocytes.

FIG. 5 is a view showing that cells, into which Tbx6, SRF and Myocd genes have been introduced, have a striated structure (bright field (BF)), and that the cells express cardiac troponin T.

FIG. 6 is a view showing the mRNA expression levels of cardiac differentiation markers, Mesp1, T, KDR, Nkx2.5 and TnnT2, in the entire cells, into which Tbx6, SRF and Myocd genes have been introduced.

FIG. 7 is a view showing the mRNA expression level of Myh11 as a marker for smooth muscle cells and the mRNA expression level of Pecam1 as a marker for vascular endothelial cells, in cells into which Tbx6, SRF and Myocd genes have been introduced.

FIG. 8 is an immunostaining fluorescence microscopic image showing cells into which Tbx6, SRF and Myocd genes have been introduced, express a smooth muscle myosin heavy chain.

### DESCRIPTION OF EMBODIMENTS

The present invention relates to a method for producing cardiac progenitor cells, comprising introducing a Tbx6 gene into fibroblasts, and fibroblast-derived cardiac progenitor cells, comprising an exogenous Tbx6 gene.

When a Tbx6 gene is introduced into fibroblasts by the use of retrovirus, cells, which express Mesp1 as a cardiac progenitor cell-specific marker, are induced. Even 30 days after introduction of Tbx6, such a cardiac progenitor cell-related gene is expressed, and the state of cardiac progenitor cells is maintained. A Tbx6 polypeptide is expressed in fibroblasts, and as a result, the Tbx6 gene-introduced fibroblasts are directly reprogrammed to differentiated cardiac progenitor cells, without going through stem cells or progenitor cells.

That is to say, the method for producing cardiac progenitor cells of the present invention comprises a step of introducing a Tbx6 gene into fibroblasts. According to the method for producing cardiac progenitor cells of the present invention, cardiac progenitor cells can be efficiently produced by introduction of only one factor. Moreover, since the expression of a marker gene in the cardiac progenitor cells is maintained even 1 month after the introduction of Tbx6, cardiac progenitor cells having proliferation ability can be stably produced according to the present invention.

Furthermore, the present invention relates to a method for producing cardiomyocytes, comprising introducing Tbx6, SRF and Myocardin (Myocd) genes into fibroblasts, and fibroblast-derived cardiomyocytes comprising an exogenous Tbx6 gene, an exogenous SRF gene and an exogenous Myocd gene.

When three factors (TSM), namely, Tbx6, and also, SRF and Myocd that are highly expressed in differentiated cardiomyocytes, are introduced into fibroblasts by the use of retrovirus, mature beating cardiomyocytes are induced. In addition, in the induced cardiomyocytes, the expression of cardiac muscle-specific genes such as Nkx2.5 or troponin, and the formation of a striated structure are confirmed. In fibroblasts, Tbx6, SRF and Myocd polypeptides are expressed, and as a result, the fibroblasts, into which the Tbx6, SRF and Myocd genes have been introduced, are directly reprogrammed to differentiated cardiomyocytes without going through stem cells or progenitor cells.

Further, when the Tbx6, SRF and Myocd genes are introduced into fibroblasts, not only the expression of cardiomyocytes-specific genes, but also the expression of cardiac progenitor cell genes (Mesp1, T, and KDR) is induced. Accordingly, since cell differentiation goes through cardiac progenitor cells according to the present invention, cardiomyocytes, smooth muscle cells, or vascular endothelial cells can be produced.

That is to say, the method for producing cardiomyocytes of the present invention comprises a step of introducing a Tbx6 gene, an SRF gene and a Myocd gene into fibroblasts. According to the method for producing cardiomyocytes of the present invention, functionally mature cardiomyocytes can be produced.

Hence, the present invention provides fibroblast-derived cardiac progenitor cells, into which a Tbx6 gene has been introduced, or fibroblast-derived cardiomyocytes, into which a Tbx6 gene, an SRF gene and a Myocd gene have been introduced. In addition, the present invention provides an inducer for inducing cardiac progenitor cells, which comprises a Tbx6 gene, or an inducer for inducing cardiomyocytes, which comprises a Tbx6 gene, an SRF gene, and a Myocd gene. Moreover, since not only cardiomyocytes, but also smooth muscle cells or endothelial cells are induced by introducing a Tbx6 gene, an SRF gene and a Myocd gene into fibroblasts, according to the present invention, an inducer for inducing smooth muscle cells, or an inducer for inducing endothelial cells, each of which comprises a Tbx6 gene, an SRF gene, and a Myocd gene, is provided.

### [Direct Reprogramming]

In the present invention, fibroblasts, into which a Tbx6 gene has been introduced, or fibroblasts, into which a Tbx6 gene, an SRF gene and a Myocd gene have been introduced, are directly reprogrammed to differentiated cardiac progenitor cells or differentiated cardiomyocytes, without going through stem cells or progenitor cells.

### [Introduction of Reprogramming Factors]

In one aspect of the present invention, a Tbx6 gene, or a set of a Tbx6 gene, an SRF gene and a Myocd gene can be introduced into fibroblasts in vitro. In addition, the fibroblasts are induced to differentiate into cardiac progenitor cells or cardiomyocytes in vitro. The induced cardiac progenitor cells or the induced cardiomyocytes can be introduced into an individual body.

In another aspect of the present invention, a Tbx6 gene, or a set of a Tbx6 gene, an SRF gene and a Myocd gene can be introduced in vivo into fibroblasts, for example, into the diseased cardiac tissues of an individual body. In addition, the fibroblasts are induced to differentiate into cardiac progenitor cells or cardiomyocytes in vivo.

In another aspect of the present invention, a Tbx6 gene, or a set of a Tbx6 gene, an SRF gene and a Myocd gene can be introduced into fibroblasts in vitro. In addition, the fibroblasts are introduced into an individual body, and are then induced to differentiate into cardiac progenitor cells or cardiomyocytes in vivo.

Introduction of a reprogramming factor gene (a Tbx6 gene, an SRF gene or a Myocd gene) into fibroblasts can be carried out by introducing a nucleic acid comprising a nucleotide sequence encoding Tbx6, a nucleic acid comprising a nucleotide sequence encoding SRF, or a nucleic acid comprising a nucleotide sequence encoding Myocd, into fibroblasts. The thus gene-introduced fibroblasts are induced to differentiate into cardiac progenitor cells or cardiomyocytes by expressing Tbx6, SRF or Myocd. Accordingly, the step of introducing a Tbx6 gene into fibroblasts may comprise introducing a Tbx6 polypeptide into fibroblasts. On the other hand, the step of introducing a Tbx6 gene, an SRF gene and a Myocd gene into fibroblasts may comprise introducing a Tbx6 polypeptide, an SRF polypeptide and a Myocd polypeptide into fibroblasts.

Moreover, the method for producing cardiac progenitor cells or cardiomyocytes of the present invention may comprise a step of transforming fibroblasts using a Tbx6 gene, or a step of transforming fibroblasts using a Tbx6 gene, an SRF gene and a Myocd gene. Furthermore, the method for producing cardiac progenitor cells or cardiomyocytes of the present invention may comprise a step of expressing a Tbx6 gene in fibroblasts, or a step of expressing a Tbx6 gene, an SRF gene and a Myocd gene in fibroblasts.

Fibroblasts, into which a Tbx6 gene has been introduced, or fibroblasts, into which a Tbx6 gene, an SRF gene and a Myocd gene have been introduced, are induced to differentiate into cardiac progenitor cells or cardiomyocytes within a certain period of time, for example, within 7 to 14 days, and preferably within 7 days. For example, when a Tbx6 gene, or a set of a Tbx6 gene, an SRF gene and a Myocd gene is introduced into a fibroblast population, at least 10%, at least 15%, at least 20%, at least 30%, at least 50%, at least 70%, or at least 90% of the population is reprogrammed to cardiac progenitor cells or cardiomyocytes, for example, within a period of 7 to 14 days, and preferably within a period of 7 days.

In the method for producing cardiac progenitor cells or cardiomyocytes of the present invention, a certain period of time (for example, 7 to 14 days, and preferably 7 days) after the step of introducing a Tbx6 gene, or a set of a Tbx6 gene, an SRF gene and a Myocd gene into fibroblasts a step of sorting a population of the fibroblasts is carried out, so that the ratio of the cardiac progenitor cells or the cardiomyocytes can be enriched. Such a sorting step is performed regarding the positive expression of a fibroblast-specific marker such as vimentin, poly-1-4-hydroxylase, a fibroblast-specific protein, a fibroblast surface antigen, or type 1 collagen, so that remaining fibroblasts can be removed if they remain. In addition, a sorting step is performed regarding the expression of a marker specific to cardiac progenitor cells or cardiomyocytes, so that the ratio of such cells can be enriched.

Moreover, when the method for producing cardiac progenitor cells or cardiomyocytes of the present invention comprises a step of introducing a nucleic acid comprising a nucleotide sequence encoding a detectable marker into fibroblasts, it can give a means for the sorting step, or a means for confirming induction of differentiation into cardiac progenitor cells or cardiomyocytes. Such a nucleotide sequence encoding a detectable marker is operably linked to a cardiac progenitor cell-specific promoter or a cardiomyocyte-specific promoter, or is linked to a nucleotide sequence encoding a cardiac progenitor cell-specific marker or a nucleotide sequence encoding a cardiomyocyte-specific marker. Examples of the detectable marker include: polypeptides directly generating detectable signals, for example, fluorescent proteins such as GFP, YEP, or BFP; and enzymes generating detectable signals when they act on a substrate, such as luciferase or alkaline phosphatase. Examples of the cardiac progenitor cell-specific promoter include promoters of Mesp1, T, or Flk1 (KDR). Examples of the promoter specific to cardiomyocytes include an α-myosin heavy chain promoter and a cTnT promoter. The expression of a detectable marker enables detection of cardiac progenitor cells or cardiomyocytes, and as a result, it can give a means for confirming induction of differentiation into cardiac progenitor cells or cardiomyocytes, or sorting the induced cardiac progenitor cells or the induced cardiomyocytes.

In the present description, the phrase “operably linking” is used to mean functional linking between nucleic acids, which gives a desired function such as transcription or translation. For example, this linking includes functional linking between a nucleic acid expression control sequence such as a promoter or a signal sequence, and a second polynucleotide. The expression control sequence has an influence on the transcription and/or translation of the second polynucleotide.

### [Fibroblasts]

In the present invention, as fibroblasts, mammalian fibroblasts, such as human fibroblasts, or the fibroblasts of mammals other than a human, such as a mouse, a rat, a swine, a monkey, a horse, a bovine, sheep, a goat or a dog, can be used. The fibroblasts are preferably human fibroblasts. In another embodiment, the fibroblasts are mouse fibroblasts. The fibroblasts may be fibroblasts obtained from mammals, or may also be progenies isolated from the fibroblasts obtained from mammals, or may further be the sub-cultured cells thereof. As such fibroblasts, for example, embryonic fibroblasts, tail tip-derived fibroblasts, cardiac fibroblasts, foreskin fibroblasts, skin fibroblasts, lung fibroblasts, etc. can be used.

A medium used in the culture of fibroblasts, such as MEM, DMEM, or IMDM medium, can be appropriately selected or prepared by a person skilled in the art. The fibroblasts can be cultured in the presence or absence of serum. The culture is not particularly limited, as long as it is carried out under conditions suitable for the culture of fibroblasts. In general, fibroblasts are cultured in a temperature range of 25° C. to 37° C. under conditions of 5% CO₂.

### [Reprogramming Factor]

In the method for producing cardiac progenitor cells or cardiomyocytes of the present invention, one or more nucleic acids comprising nucleotide sequences encoding one or more reprogramming factors can be introduced into fibroblasts. Otherwise, in the method for producing cardiac progenitor cells or cardiomyocytes of the present invention, one or more reprogramming factor polypeptides themselves can be introduced into fibroblasts. In the present invention, when cardiac progenitor cells are to be produced, the reprogramming factor is Tbx6. When cardiomyocytes are to be produced, the reprogramming factors are Tbx6, SRF, and Myocd (TSM). Tbx6, SRF, and Myocd can be introduced, at once or successively, into cells. The phrase “introduction into cells at once” means that a plurality of reprogramming factors are introduced into cells by a single cell introduction step. On the other hand, the phrase “introduction into cells successively” means that a plurality of reprogramming factors are introduced into cells by a plurality of cell introduction steps on the same day or on the different days. From the viewpoint of cell introduction efficiency, it is preferable to introduce a plurality of reprogramming factors into cells at once. In addition, the amino acid sequences of the reprogramming factors of the present invention, and nucleotide sequences encoding the amino acid sequences are known in the present technical field.

### [Tbx6]

A Tbx6 polypeptide (T-box transcriptional factor 6) is a transcriptional factor that binds to T box in the promoter region of a certain gene and recognizes it. The amino acid sequences of Tbx6 polypeptides derived from various species, and nucleotide sequences encoding the Tbx6 polypeptides derived from various species have been known. For example, Genbank Accession Nos. NM_004608.3 (human, nucleotide sequence, SEQ ID NO: 1, CDS 61 . . . 1371), NP 004599.2 (human, amino acid sequence, SEQ ID NO: 2), NM_011538.2 (mouse, nucleotide sequence, SEQ ID NO: 3, CDS 25 . . . 1335), NP_035668.2 (mouse, amino acid sequence, SEQ ID NO: 4), and the like may be referred to.

Moreover, in one aspect of the present invention, the Tbx6 polypeptide includes a polypeptide, which has an amino acid sequence having a sequence identity of at least 80%, 85%, 90%, 91%, 92%, 93%, 94%, 95%, 96%, 97%, 98%, 99% or 100% to the amino acid sequence shown in SEQ ID NO: 2 or SEQ ID NO: 4, and has a function of inducing fibroblasts to differentiate into cardiac progenitor cells, when it is introduced into the fibroblasts.

Furthermore, in one aspect of the present invention, the Tbx6 polypeptide includes a polypeptide, which has an amino acid sequence comprising a deletion, substitution, insertion or addition of 1 to 50, preferably 1 to 40, more preferably 1 to 20, and further preferably 1 to 10 (for example, 1, 2, 3, 4, 5, 6, 7, 8, 9 or 10) amino acids, or a combination thereof, with respect to the amino acid sequence shown in SEQ ID NO: 2 or SEQ ID NO: 4, and has a function of inducing fibroblasts to differentiate into cardiac progenitor cells, when it is introduced into the fibroblasts.

Further, in one aspect of the present invention, the Tbx6 polypeptide includes a polypeptide, which has an amino acid sequence encoded by a nucleotide sequence having a sequence identity of at least 80%, 85%, 90%, 91%, 92%, 93%, 94%, 95%, 96%, 97%, 98%, 99% or 100% to the nucleotide sequence shown in SEQ ID NO: 1 or SEQ ID NO: 3, and has a function of inducing fibroblasts to differentiate into cardiac progenitor cells, when it is introduced into the fibroblasts.

Still further, in one aspect of the present invention, the Tbx6 gene (nucleic acid) includes a nucleic acid, which has a nucleotide sequence having a sequence identity of at least 80%, 85%, 90%, 91%, 92%, 93%, 94%, 95%, 96%, 97%, 98%, 99% or 100% to the nucleotide sequence shown in SEQ ID NO: 1 or SEQ ID NO: 3, and has a function of inducing fibroblasts to differentiate into cardiac progenitor cells, when the polypeptide encoded by the nucleic acid is introduced into the fibroblasts.

### [SRF]

The amino acid sequences of SRF polypeptides derived from various species, and nucleotide sequences encoding the SRF polypeptides derived from various species have been known. For example, Genbank Accession Nos. NM_003131.3 (human, nucleotide sequence, SEQ ID NO: 5, CDS 363 . . . 1889), NP_003122.1 (human, amino acid sequence, SEQ ID NO: 6), NM_020493.2 (mouse, nucleotide sequence, SEQ ID NO: 7, CDS 335 . . . 1849), NP_065239.1 (mouse, amino acid sequence, SEQ ID NO: 8), and the like may be referred to.

Moreover, in one aspect of the present invention, the SRF polypeptide includes a polypeptide, which has an amino acid sequence having a sequence identity of at least 80%, 85%, 90%, 91%, 92%, 93%, 94%, 95%, 96%, 97%, 98%, 99% or 100% to the amino acid sequence shown in SEQ ID NO: 6 or SEQ ID NO: 8, and has a function of inducing fibroblasts to differentiate into cardiomyocytes, when it is introduced, together with Tbx6 and Myocardin, into the fibroblasts.

Furthermore, in one aspect of the present invention, the SRF polypeptide includes a polypeptide, which has an amino acid sequence comprising a deletion, substitution, insertion or addition of 1 to 60, preferably 1 to 50, more preferably 1 to 25, and further preferably 1 to 13 (for example, 1, 2, 3, 4, 5, 6, 7, 8, 9, 10, 11, 12 or 13) amino acids, or a combination thereof, with respect to the amino acid sequence shown in SEQ ID NO: 6 or SEQ ID NO: 8, and has a function of inducing fibroblasts to differentiate into cardiomyocytes, when it is introduced, together with Tbx6 and Myocardin, into the fibroblasts.

Further, in one aspect of the present invention, the SRF polypeptide includes a polypeptide, which has an amino acid sequence encoded by a nucleotide sequence having a sequence identity of at least 80%, 85%, 90%, 91%, 92%, 93%, 94%, 95%, 96%, 97%, 98%, 99% or 100% to the nucleotide sequence shown in SEQ ID NO: 5 or SEQ ID NO: 7, and has a function of inducing fibroblasts to differentiate into cardiomyocytes, when it is introduced, together with Tbx6 and Myocardin, into the fibroblasts.

Still further, in one aspect of the present invention, the SRF nucleic acid includes a nucleic acid, which has a nucleotide sequence having a sequence identity of at least 80%, 85%, 90%, 91%, 92%, 93%, 94%, 95%, 96%, 97%, 98%, 99% or 100% to the nucleotide sequence shown in SEQ ID NO: 5 or SEQ ID NO: 7, and has a function of inducing fibroblasts to differentiate into cardiomyocytes, when it is introduced, together with Tbx6 and Myocardin, into the fibroblasts.

### [Myocardin] (Myocd)

The amino acid sequences of Myocardin polypeptides derived from various species, and nucleotide sequences encoding the Myocardin polypeptides derived from various species have been known. For example, Genbank Accession Nos. NM_001146312.2 (human, nucleotide sequence, SEQ ID NO: 9, CDS 300 . . . 3260), NP_001139784.1 (human, amino acid sequence, SEQ ID NO: 10), NM_145136.4 (mouse, nucleotide sequence, SEQ ID NO: 11, CDS 292 . . . 3243), NP_660118.3 (mouse, amino acid sequence, SEQ ID NO: 12), and the like may be referred to.

Moreover, in one aspect of the present invention, the Myocardin polypeptide includes a polypeptide, which has an amino acid sequence having a sequence identity of at least 80%, 85%, 90%, 91%, 92%, 93%, 94%, 95%, 96%, 97%, 98%, 99% or 100% to the amino acid sequence shown in SEQ ID NO: 10 or SEQ ID NO: 12, and has a function of inducing fibroblasts to differentiate into cardiomyocytes, when it is introduced, together with Tbx6 and SRF, into the fibroblasts.

Furthermore, in one aspect of the present invention, the Myocardin polypeptide includes a polypeptide, which has an amino acid sequence comprising a deletion, substitution, insertion or addition of 1 to 100, preferably 1 to 50, more preferably 1 to 30, and further preferably 1 to 20 (for example, 1, 2, 3, 4, 5, 6, 7, 8, 9, 10, 11, 12, 13, 14, 15, 16, 17, 18, 19 or 20) amino acids, or a combination thereof, with respect to the amino acid sequence shown in SEQ ID NO: 10 or SEQ ID NO: 12, and has a function of inducing fibroblasts to differentiate into cardiomyocytes, when it is introduced, together with Tbx6 and SRF, into the fibroblasts.

Further, in one aspect of the present invention, the Myocardin polypeptide includes a polypeptide, which has an amino acid sequence encoded by a nucleotide sequence having a sequence identity of at least 80%, 85%, 90%, 91%, 92%, 93%, 94%, 95%, 96%, 97%, 98%, 99% or 100% to the nucleotide sequence shown in SEQ ID NO: 9 or SEQ ID NO: 11, and has a function of inducing fibroblasts to differentiate into cardiomyocytes, when it is introduced, together with Tbx6 and SRF, into the fibroblasts.

Still further, in one aspect of the present invention, the Myocardin gene (nucleic acid) includes a nucleic acid, which has a nucleotide sequence having a sequence identity of at least 80%, 85%, 90%, 91%, 92%, 93%, 94%, 95%, 96%, 97%, 98%, 99% or 100% to the nucleotide sequence shown in SEQ ID NO: 9 or SEQ ID NO: 11, and has a function of inducing fibroblasts to differentiate into cardiomyocytes, when it is introduced, together with Tbx6 and SRF, into the fibroblasts.

To date, it has been reported that functionally immature cardiomyocyte-like cells can be produced from fibroblasts, through cardiac progenitor cells, by using transcriptional factors (Mesp1 and Ets2) and a plurality of humoral factors (Non Patent Literature 2). In the present invention, such transcriptional factors do not need to be introduced into fibroblasts, and also, such transcriptional factors do not need to be combined with humoral factors.

### [Cardiac Progenitor Cells]

In the present invention, the “cardiac progenitor cells” are characterized in that the cells express markers specific to the cardiac progenitor cells. The markers specific to cardiac progenitor cells are factors that are specifically expressed in cardiac progenitor cells (cardiac progenitor cell-related factors). Examples of the marker specific to cardiac progenitor cells include T, Mesp1, Flk1 (KDR), Pdgfrα, and Is11. Cardiac progenitor cells express at least one, more preferably at least two, and further preferably at least three of such cardiac progenitor cell-specific markers. Cardiac progenitor cells are preferably cells, which express T, Mesp1, and Flk1 (KDR). The expression of a marker can be confirmed at a gene level or a protein level.

In addition, in the present invention, since cardiac progenitor cells are induced from fibroblasts, the cardiac progenitor cells may also be referred to as “induced cardiac progenitor cells.”

### [Cardiomyocytes]

In the present invention, the “cardiomyocytes” are characterized in that the cells express markers specific to the cardiomyocytes. The markers specific to cardiomyocytes are factors that are specifically expressed in cardiomyocytes (myocardial cell-related factors). Examples of the marker specific to cardiomyocytes include cardiac troponin (cTnT), Nkx2.5, and Actn2. Cardiomyocytes express at least one, more preferably at least two, and further preferably at least three of such cardiomyocyte-specific markers. Cardiomyocytes are preferably cells, which express cTnT and Nkx2.5.

Moreover, in the present invention, the “cardiomyocytes” may be characterized in that the cells beat. Furthermore, in the present invention, the “cardiomyocytes” may also be characterized in that the cells form a striated structure.

Further, in the present invention, since cardiomyocytes are induced from fibroblasts, the cardiomyocytes may also be referred to as “induced cardiomyocytes.”

The expression of various markers specific to cardiac progenitor cells or cardiomyocytes can be detected by biochemical or immunochemical approaches (for example, an enzyme-linked immunosorbent assay, an immunohistochemical assay, etc.). Alternatively, the expression of such markers can also be detected by measuring the expression of nucleic acids encoding various markers specific to cardiac progenitor cells or cardiomyocytes. The expression of such nucleic acids encoding various markers specific to cardiac progenitor cells or cardiomyocytes can be confirmed by molecular biological approaches such as RT-PCR or hybridization. Primers or probes used in these approaches can be appropriately designed and produced by a person skilled in the art, using information available from database such as Genbank.

The beating of cardiomyocytes can be confirmed by visual observation or by observing the bright field image thereof. In addition, it is also possible to confirm spontaneous contraction by standard electrophysiological methods such as a patch clamp method.

Moreover, the formation of a striated structure by cardiomyocytes can be confirmed by visual observation or by observing the bright field image thereof. Furthermore, it can also be confirmed by performing immunostaining on proteins contributing to a cardiac muscle structure, such as troponin.

[Introduction of Exogenous Reprogramming Factor Polypeptides into Fibroblasts]

In the present description, the term “exogenous” means a nucleic acid or a polypeptide to be introduced into certain cells (for example, by electroporation, infection, lipofection, microinjection, or any other methods of introducing a nucleic acid into cells).

In one aspect of the present invention, the exogenous reprogramming factors can also be introduced into fibroblasts by allowing the polypeptides of Tbx6, SRF and/or Myocd to come into contact with the fibroblasts. The polypeptides of Tbx6, SRF, and Myocd can be each produced by a genetically engineering method or a molecular biological method, based on information regarding amino acid sequences and nucleotide sequences stored in known database.

[Introduction of Exogenous Reprogramming Factor Genes into Fibroblasts]

Introduction of the exogenous reprogramming factor (Tbx6, SRF, and Myocd) genes, namely, introduction of polynucleotides encoding these polypeptides or polynucleotides having nucleotide sequences complementary to the nucleotide sequences thereof, can be carried out by known transformation methods, such as viral infection using, for example, viral vectors such as a retroviral vector or an adenoviral vector, a lipofection method, an electroporation method, a microinjection method, or a calcium phosphate method.

In another aspect of the present invention, introduction of exogenous reprogramming factor polypeptides (Tbx6, or a set of Tbx6, SRF and Myocd) into fibroblasts is achieved by introducing exogenous nucleic acids comprising nucleotide sequences encoding the reprogramming factor polypeptides into fibroblasts. The species as origins of the exogenous reprogramming factors are preferably identical to the species as origins of the fibroblasts, such as, for example, a human and a human, or a mouse and a mouse.

In the present invention, an exogenous nucleic acid comprising a nucleotide sequence encoding an exogenous reprogramming factor polypeptide can be in the form of a recombinant expression vector comprising an expression cassette. In such a case, examples of a suitable vector include: recombinant retrovirus, lentivirus, and adenovirus; and a retrovirus expression vector, a lentivirus expression vector, a nucleic acid expression vector, and a plasmid expression vector. In another aspect of the present invention, an exogenous nucleic acid is incorporated into the genome of fibroblasts and the progenies thereof.

In an aspect of the present invention, fibroblasts are transformed with different expression constructs (expression vectors) each comprising a nucleotide sequence encoding Tbx6, SRF, or Myocd. In another aspect of the present invention, the expression construct may comprise nucleotide sequences encoding two or more of Tbx6, SRF, and Myocd. In an aspect of the present invention, the expression construct comprises nucleotide sequences encoding Tbx6, SRF, and Myocd.

In the present invention, an exogenous nucleic acid comprising a nucleotide sequence encoding the Tbx6 polypeptide, or an exogenous nucleic acid(s) comprising a nucleotide sequence(s) encoding one or more of Tbx6, SRF and Myocd polypeptides, is (are) introduced in vitro into a single fibroblast or a population of fibroblasts, or is (are) introduced in vivo in a single fibroblast or a population of fibroblasts.

In another aspect of the present invention, a nucleic acid(s) comprising a nucleotide sequence(s) encoding the Tbx6 polypeptide, or one or more of the Tbx6, SRF and Myocd polypeptides, may be an expression construct(s) capable of production of the reprogramming factor polypeptide(s) in fibroblasts. In another aspect of the present invention, examples of the expression construct include viral constructs such as a recombinant adeno-associated viral construct (see, for example, U.S. Pat. No. 7,078,387), a recombinant adenoviral construct, and a recombinant lentiviral construct.

Examples of a suitable expression vector include viral vectors (e.g., vaccinia virus-based viral vectors; polio virus; adenovirus (see, for example, Li et al., Invest Opthalmol Vis Sci 35: 2543 2549, 1994; Borras et al., Gene Ther 6: 515 524, 1999; Li and Davidson, PNAS 92: 7700 7704, 1995; Sakamoto et al., H Gene Ther 5: 1088 1097, 1999; International Publication WO No. 94/12649; International Publication WO No. 93/03769; International Publication WO No. 93/19191; International Publication WO No. 94/28938; International Publication WO No. 95/11984; and International Publication WO No. 95/00655); adeno-associated virus (see, for example, Ali et al., Hum Gene Ther 9: 81 86, 1998, Flannery et al., PNAS 94: 6916 6921, 1997; Bennett et al., Invest Opthalmol Vis Sci 38: 2857-2863, 1997; Jomary et al., Gene Ther4: 683-690, 1997, Rolling et al., Hum Gene Ther 10: 641 648, 1999; Ali et al., Hum Mol Genet 5: 591-594, 1996; Srivastava, International Publication WO No. 93/09239, Samulski et al., J. Vir. (1989) 63: 3822 3828; Mendelson et al., Virol. (1988) 166: 154 165; and Flotte et al., PNAS (1993) 90: 10613-10617); SV40; herpes simplex virus; human immunodeficiency virus (see, for example, Miyoshi et al., PNAS94: 10319-23, 1997; Takahashi et al., J Virol 73: 7812 7816, 1999); retroviral vectors (e.g., vectors derived from murine leukemia virus, spleen necrosis virus, and retrovirus, for example, Rous sarcomere virus, Harvey sarcomere virus, avian leukemia virus, lentivirus, human immunodeficiency virus, myeloproliferative sarcomere virus, and breast cancer virus), but the examples of a suitable expression vector are not limited thereto.

A large number of suitable expression vectors have been known in the present technical field, and many expression vectors are commercially available. The below-mentioned vectors are presented for illustrative purposes, and for eukaryotic host cells, pXT1, pSGS (Stratagene), pSVK3, pBPV, pMSG, and pSVLSV40 (Pharmacia) are used. However, any other vectors can also be used as long as they are compatible with host cells.

Depending on the used host/vector system, any of many suitable transcriptional and translational regulatory elements, such as constitutive and inducible promoters, transcriptional enhancer elements, and transcriptional terminators, may be used in an expression vector (see, for example, BITTER et al. (1987) METHODS IN ENZYMOLOGY, 153: 516-544).

In another aspect of the present invention, nucleotide sequences encoding reprogramming factors (e.g., Tbx6 CDR sequence, SRF CDR sequence, and Myocd CDR sequence) may be operably linked to regulatory elements, for example, to transcriptional regulatory elements, such as promoters. The transcriptional regulatory elements function in eukaryotic cells, such as in mammalian cells. Suitable transcriptional regulatory elements include promoters and enhancers. In another aspect of the present invention, promoters are constitutively active. In another embodiment, promoters are inducible.

Non-restrictive examples of a suitable eukaryotic promoter (a promoter functioning in eukaryotic cells) include CMV immediate early, HSV thymidine kinase, early and late SV40, retrovirus-derived long terminal repeat (LTR), and mouse metallothionein-I.

In another aspect of the present invention, a nucleotide sequence encoding a reprogramming factor is operably linked to heart-specific transcriptional regulatory elements (TRE), and in such a case, TRE may include promoters and enhancers. Suitable TRE includes TRE derived from the below-mentioned genes, namely, from myosin light chain-2, α-myosin heavy chain, AE3, cardiac troponin C, and cardiac actin, but the examples of the TRE are not limited thereto (Franz et al. (1997) Cardiovasc. Res. 35: 560-566; Robbins et al. (1995) Ann. N. Y. Acad. Sci. 752: 492-505; Linn et al. (1995) Circ. Res. 76: 584-591; Parmacek et al. (1994) Mol. Cell. Biol. 14: 1870-1885; Hunter et al. (1993) Hypertension 22:608-617; and Sartorelli et al. (1992) Proc. Natl. Acad. Sci. USA 89: 4047-4051.).

Selection of a suitable vector and a suitable promoter is well known in the present technical field. The expression vector may comprise a ribosome binding site for initiation of translation and initiation of transcription. The expression vector may comprise a suitable sequence for amplifying the expression.

Examples of a suitable mammalian expression vector (an expression vector suitable for use in mammalian host cells) include recombinant virus, nucleic acid vectors, such as a plasmid, a bacterial artificial chromosome, a yeast artificial chromosome, a human artificial chromosome, cDNA, cRNA, and a polymerase chain reaction (PCR) product expression cassette, but the examples of a suitable mammalian expression vector are not limited thereto. Examples of a suitable promoter for driving the expression of nucleotide sequences encoding Tbx6, SRF, and Myocd include retrovirus long terminal repeat (LTR) element; constitutive promoters such as CMV, HSV1-TK, SV40, EF-1α, or β-actin; and phosphoglycerol kinase (PGK), and inducible promoters, such as those containing a Tet-operator element, but the examples of the suitable promoter are not limited thereto. In some cases, such a mammalian expression vector may encode a marker gene that facilitates discrimination or selection of transfected or infected cells, as well as exogenous Tbx6, SRF and Myocd polypeptides. Examples of the marker gene include: genes encoding fluorescent proteins such as an enhanced green fluorescent protein, Ds-Red (DsRed: Discosomasp. red fluorescent protein (RFP); Bevis and Glick (2002) Nat. Biotechnol. 20: 83), a yellow fluorescent protein, and a cyan fluorescent protein; and genes encoding proteins that impart resistance to selective agents, such as a neomycin resistance gene, a puromycin resistance gene, and a blasticidin resistance gene, but the examples of the marker gene are not limited thereto.

Examples of a suitable viral vector include: a retrovirus-based viral vector (including lentivirus); adenovirus; and adeno-associated virus, but the examples are not limited thereto. A suitable retrovirus-based vector is Moloney murine leukemia virus (MMLV)-based vector, but other recombinant retroviruses may also be used. Examples of such other recombinant retroviruses include avian leukemia virus, bovine leukemia virus, murine leukemia virus (MLV), mink cell focus inducing virus, murine sarcomere virus, reticuloendotheliosis virus, gibbon ape leukemia virus, Mason Pfizer monkey virus, and Rous sarcomere virus. For instance, U.S. Pat. No. 6,333,195 can be referred to.

In another case, the retrovirus-based vector may be a lentivirus-based vector (e.g., human immunodeficiency virus-1 (HIV-1); simian immunodeficiency virus (SIV); or ferine immunodeficiency virus (FIV)), and for instance, Johnston et al. (1999), Journal of Virology, 73(6): 4991-5000 (FIV); Negre D et al. (2002), Current Topics in Microbiology and Immunology, 261: 53-74(SIV); and Naldini et al. (1996), Science, 272: 263-267 (HIV) can be referred to.

In order to support incorporation of recombinant retrovirus into target cells, such recombinant retrovirus may comprise a viral polypeptide (e.g., retrovirus env). Such a viral polypeptide has been sufficiently established in the present technical field, and for instance, U.S. Pat. No. 5,449,614 can be referred to. The viral polypeptide may be an amphotropic viral polypeptide, for example, amphotropic env. Such an amphotropic viral polypeptide supports incorporation of retrovirus into cells derived from a large number of species including cells that are out of the original host species. The viral polypeptide may be a xenotropic viral polypeptide supporting incorporation of retrovirus into cells that are out of the original host species. In another aspect of the present invention, the viral polypeptide is an ecotropic virus polypeptide, for example, ecotropic env, and such an ecotropic virus polypeptide supports incorporation of retrovirus into the cells of the original host species.

Examples of the viral polypeptide capable of supporting incorporation of retrovirus into cells include MMLV amphotropic env, MMLV ecotropic env, MMLV xenotropic env, vesicular stomatitis virus-g protein (VSV-g), HIV-1 env, gibbon ape leukemia virus (GALV) env, RD114, FeLV-C, FeLV-B, MLV10A1 env gene, and mutants thereof, such as chimera, but the examples are not limited thereto. For instance, Yee et al. (1994), Methods Cell Biol., PtA: 99-112(VSV-G); and U.S. Pat. No. 5,449,614 can be referred to. In some cases, in order to promote expression or reinforced binding to a receptor, the viral polypeptide is genetically modified.

In general, recombinant virus is produced by introducing a viral DNA or RNA construct into producer cells. In some cases, the producer cells do not express an exogenous gene. In other cases, the producer cells are “packaging cells” comprising one or more exogenous genes, for example, genes encoding one or more of gag, pol, or env polypeptide, and/or one or more of retrovirus gag, pol, or env polypeptide. Retrovirus packaging cells may comprise a gene encoding a viral polypeptide, for example, VSV-g that supports incorporation of retrovirus into target cells. In some cases, the packaging cells comprise genes encoding one or more lentivirus proteins, such as gag, pol, env, vpr, vpu, vpx, vif, tat, rev, or nef. In some cases, the packaging cells comprise genes encoding adenovirus proteins, such as E1A or E1B, or other adenovirus proteins. For instance, proteins supplied by such packaging cells may be: retrovirus-derived proteins, such as gag, pol, and env; lentivirus-derived proteins, such as gag, pol, env, vpr, vpu, vpx, vif, tat, rev, and nef; and adenovirus-derived proteins, such as E1A and E1B. In many examples, the packaging cells supply proteins derived from viruses that are different from the virus as an origin of the viral vector.

Examples of the packaging cell line include cell lines that can be easily transfected, but the examples are not limited thereto. The packaging cell line can be based on 293T cells, NIH3T3, COS, or HeLa cell lines. The packaging cells are frequently used for packaging a viral vector plasmid comprising a deletion of at least one gene encoding a protein necessary for virus packaging. Cells capable of supplying a deleted protein or polypeptide from a protein encoded by such a viral vector plasmid may be used as packaging cells. Examples of the packaging cell line include Platinum-E (Plat-E); Platinum-A (Plat-A); BOSC23 (ATCCCRL11554); and Bing (ATCC CRL 11270), but are not limited thereto. For instance, Morita et al. (2000), Gene Therapy, 7: 1063-1066; Onishi et al. (1996), Experimental Hematology, 24: 324-329; and U.S. Pat. No. 6,995,009 can be referred to. Commercially available packaging lines are also useful, and examples of such a commercially available packaging line include Ampho-Pak293 cell line, Eco-Pak2-293 cell line, RetroPackPT67 cell line, and Retro-X Universal Packaging System (all of which are available from Clontech).

The retroviral construct may be derived from a certain range of retroviruses, such as MMLV, HIV-1, SIV or FIV, or from other retroviruses described in the present description. The retroviral construct may encode all viral polypeptides necessary for one or more replication cycles of a specific virus. In some cases, the efficiency of incorporation of virus is improved by addition of other factors or other viral polypeptides. In other cases, as described in U.S. Pat. No. 6,872,528, a viral polypeptide encoded by a retroviral construct does not support more than one cycle of replication. Under such circumstances, promotion of virus incorporation can be supported by addition of other factors or other viral polypeptides. In the illustrative embodiment, the recombinant retrovirus is an HIV-1 virus, which comprises a VSV-g polypeptide but does not comprise an HIV-1 env polypeptide.

The retroviral construct may comprise a promoter, a multicloning site, and/or a resistance gene. Examples of the promoter include CMV, SV40, EF1α, β-actin; retrovirus LTR promoter, and an inducible promoter, but the examples are not limited thereto. The retroviral construct may also comprise a packaging signal (e.g., a packaging signal derived from an MFG vector; psi packaging signal). Examples of some retroviral constructs known in the present technical field include pMX, pBabeX, and derivatives thereof, but the examples are not limited thereto. For instance, Onishi et al. (1996), Experimental Hematology, 24: 324-329 can be referred to. In some cases, the retroviral construct is a self-inactivating lentiviral vector (SIN), and for instance, Miyoshi et al. (1998), J. Virol., 72(10): 8150-8157 can be referred to. In some cases, the retroviral construct is LL-CG, LS-CG, CL-CG, CS-CG, CLG, or MFG. Miyoshi et al. (1998), J. Virol., 72(10): 8150-8157; Onishi et al. (1996), Experimental Hematology, 24: 324-329; Riviere et al. (1995), PNAS, 92: 6733-6737 can be referred to. Examples of the viral vector plasmid (or construct) include: retrovirus-based vectors, such as pMXs, pMxs-IB, pMXs-puro, and pMXs-neo (wherein pMXs-IB is a vector that supports a blasticidin resistance gene, instead of a puromycin resistance gene supported by pMXs-puro; Kimatura et al. (2003), Experimental Hematology, 31: 1007-1014; MFG Riviere et al. (1995), Proc. Natl. Acad. Sci. U.S.A., 92: 6733-6737; pBabePuro; Morgenstern et al. (1990), Nucleic Acids Research, 18: 3587-3596; LL-CG, CL-CG, CS-CG, CLG Miyoshi et al. (1998), Journal of Virology, 72: 8150-8157, etc.); and adenovirus-based vectors, such as pAdex1 (Kanegae et al. (1995), Nucleic Acids Research, 23: 3816-3821, etc.). In the illustrative embodiment, the retroviral construct comprises blasticidin (e.g., pMXs-IB), puromycin (e.g., pMXs-puro and pBabePuro); or neomycin (e.g., pMXs-neo). For instance, Morgenstern et al. (1990), Nucleic Acids Research, 8: 3587-3596 can be referred to.

Methods for producing recombinant virus from packaging cells and the use thereof have been sufficiently established; and for instance, U.S. Pat. Nos. 5,834,256; 6,910,434; 5,591,624; 5,817,491; 7,070,994; and 6,995,009 can be referred to. A majority of methods start with introduction of a viral construct into a packaging cell line. Introduction of such a viral construct includes a calcium phosphate method, a lipofection method (Felgner et al. (1987) Proc. Natl. Acad. Sci. U.S.A. 84: 7413-7417), an electroporation method, a microinjection method, FuGENE Transfection, etc., and any method described in the present description, but is not limited thereto. The viral construct may be introduced into host fibroblasts by any method known in the present technical field.

A nucleic acid construct may be introduced into host cells by applying various well-known methods, such as non-viral transfection of cells. In the illustrative aspect, the construct is incorporated into a vector, and is then introduced into host cells. Introduction of the construct into cells includes electroporation, calcium phosphate-mediated transition, nucleofection, sonoporation, heat shock, magnetofection, liposome-mediated transition, microinjection, microprojectile-mediated transition (nanoparticles), cationic polymer-mediated transition (DEAE dextran, polyethyleneimine, polyethylene glycol (PEG), etc.), and cell fusion, but is not limited thereto. Introduction of the construct into cells may be carried out by any non-viral transfection known in the present technical field. Other examples of transfection include transfection reagents, such as Lipofectamine, Dojindo Hilymax, Fugene, jetPEI, Effectene, and DreamFect.

### [Fibroblasts Comprising Exogenous Gene(s)]

The present invention includes fibroblasts comprising an exogenous Tbx6 gene. In addition, the present invention includes fibroblasts comprising an exogenous Tbx6 gene, an exogenous SRF gene, and an exogenous Myocd gene. In another aspect of the present invention, the fibroblasts comprising an exogenous gene(s) of the present invention are in an in vitro state. In another aspect of the present invention, the fibroblasts comprising an exogenous gene(s) of the present invention are mammalian cells, such as human cells, or are induced from human cells.

### [Fibroblast-Derived Cardiac Progenitor Cells or Fibroblast-Derived Cardiomyocytes Comprising Exogenous Gene(s)]

The present invention further relates to fibroblast-derived cardiac progenitor cells (induced cardiac progenitor cells, cardiac progenitor cell-like cells) or fibroblast-derived cardiomyocytes (induced cardiomyocytes, cardiomyocyte-like cells), which are produced by the above-described method for producing cardiac progenitor cells or cardiomyocytes. In the present invention, “fibroblast-derived” cardiac progenitor cells or cardiomyocytes mean cardiac progenitor cells or cardiomyocytes, which are induced from fibroblasts. Since the induced cardiac progenitor cells or induced cardiomyocytes of the present invention are induced from fibroblasts comprising an exogenous gene(s), the induced cardiac progenitor cells or induced cardiomyocytes of the present invention also comprise an exogenous Tbx6 gene, or an exogenous Tbx6 gene, an exogenous SRF gene and an exogenous Myocd gene. In another aspect of the present invention, the induced cardiac progenitor cells or induced cardiomyocytes of the present invention are in an in vitro state. In another aspect of the present invention, the induced cardiac progenitor cells or induced cardiomyocytes of the present invention are mammalian cells such as human cells, or are derived from mammalian cells such as human cells.

As described above, whether or not the cells induced from fibroblasts are cardiac progenitor cells can be confirmed using the expression of a marker specific to cardiac progenitor cells. The cells, in which the expression of a cardiac progenitor cell-specific marker has been confirmed, are also referred to as “cardiac progenitor cell-like cells.”

Likewise, as described above, whether or not the cells induced from fibroblasts are cardiomyocytes can be confirmed using the expression of a marker specific to cardiomyocytes. The cells, in which the expression of a cardiac progenitor cell-specific marker has been confirmed, are also referred to as “cardiomyocyte-like cells.”

Marker expression can be confirmed at a gene level or at a protein level.

After the induced cardiac progenitor cells of the present invention have been induced from fibroblasts, the cells can be maintained as cardiac progenitor cells for a certain period of time or longer. That is to say, the cardiac progenitor cells induced by the method of the present invention can be characterized in that, after introduction of a Tbx6 gene into the fibroblasts, the induced cardiac progenitor cells express cardiac progenitor cell-specific markers, such as T, Mesp1, or Flk1 (KDR) for a certain period of time (e.g., for 3 weeks, 4 weeks, or 5 weeks) or longer.

The present invention also provides a composition comprising fibroblasts comprising an exogenous gene(s), or fibroblast-derived cardiac progenitor cells or fibroblast-derived cardiomyocytes comprising an exogenous gene(s). The composition of the present invention comprises the above-described fibroblasts, or induced cardiac progenitor cells or induced cardiomyocytes, and may further comprise, as suitable components, salts; a buffer; a stabilizer; a protease inhibitor; a cell membrane and/or cell wall preserving compound, such as glycerol or dimethyl sulfoxide; a nutrient medium suitable for cells; and the like.

### [Inducer]

The present invention also provides an inducer for inducing cardiac progenitor cells from fibroblasts, or an inducer for inducing cardiomyocytes from fibroblasts. Moreover, since smooth muscle cells or endothelial cells are also induced by introducing a Tbx6 gene, an SRF gene and a Myocd gene into fibroblasts, the present invention also provides an inducer for inducing smooth muscle cells from fibroblasts, or an inducer for inducing vascular endothelial cells from fibroblasts.

In another aspect of the present invention, the inducer for inducing cardiac progenitor cells of the present invention comprises, at least, either 1) a Tbx6 polypeptide or 2) a nucleic acid comprising a nucleotide sequence encoding the Tbx6 polypeptide. In another aspect of the present invention, the inducer for inducing cardiomyocytes, inducer for inducing smooth muscle cells, or inducer for inducing vascular endothelial cells of the present invention comprises, at least, either 1) a mixture of a Tbx6 polypeptide, an SRF polypeptide and a Myocd polypeptide, or 2) a mixture of a nucleic acid comprising a nucleotide sequence encoding a Tbx6 polypeptide, a nucleic acid comprising a nucleotide sequence encoding an SRF polypeptide, and a nucleic acid comprising a nucleotide sequence encoding a Myocd polypeptide.

The inducer of the present invention may comprise one or more selected from: salts such as NaCl, MgCl, KCl, or MgSO₄; buffers such as a Tris buffer, N-(2-hydroxyethyl)piperazine-N′-2-ethanesulfonic acid (HEPES), 2-(N-morpholino)ethanesulfonic acid (MES), 2-(N-morpholino)ethanesulfonic acid sodium salt (MES), 3-(N-morpholino)propanesulfonic acid (MOPS), or N-tris[hydroxymethyl]methyl-3-aminopropanesulfonic acid (TAPS); solubilizers; detergents including nonionic detergents such as Tween-20; protease inhibitors; glycerol; etc., in addition to the above-described polypeptides or nucleic acids. Moreover, the inducer of the present invention may comprise a reagent for introducing the polypeptides or nucleic acids of reprogramming factors into fibroblasts.

The inducer of the present invention may be directly administered to an individual body (e.g., into cardiac tissues). The inducer of the present invention is useful for inducing fibroblasts to cardiac progenitor cells or cardiomyocytes, smooth muscle cells, or vascular endothelial cells, and this induction can be carried out in vitro or in vivo. Induction of fibroblasts to cardiac progenitor cells or cardiomyocytes can be used to treat various heart failures.

Accordingly, the inducer of the present invention may comprise a pharmaceutically acceptable excipient. Examples of a suitable excipient include water, saline, dextrose, glycerol, ethanol, and a combination thereof. Moreover, as desired, the present inducer may comprise a small amount of auxiliary substance, such as a wettable powder, an emulsifier, or a buffer. Actual methods for preparing such dosage forms have been known. For instance, Remington's Pharmaceutical Sciences, Mack Publishing Company, Easton, Pa., 17th edition, 1985 can be referred to.

A pharmaceutically acceptable excipient, such as a vehicle, an adjuvant, a carrier or a diluent, can be easily obtained. Further, a pharmaceutically acceptable auxiliary substance, such as a pH adjuster, a buffer, a tension adjuster, a stabilizer or a wettable powder, can be easily purchased.

### [Therapeutic Method Using Cells]

The fibroblasts comprising an exogenous gene(s) of the present invention can be used to treat an individual in need of the treatment. Likewise, the induced cardiac progenitor cells or induced cardiomyocytes of the present invention can be used to treat an individual in need of the treatment. The fibroblasts comprising an exogenous gene(s) of the present invention, or the induced cardiac progenitor cells or induced cardiomyocytes of the present invention, can be introduced into a recipient individual (an individual in need of treatment), and in such a case, introduction of the fibroblasts comprising an exogenous gene(s) of the present invention, or the induced cardiac progenitor cells or induced cardiomyocytes of the present invention, into such a recipient individual provides the treatment of the condition or disorder of the individual. Therefore, the present invention relates to a therapeutic method comprising administering the fibroblasts comprising an exogenous gene(s) of the present invention, or the induced cardiac progenitor cells or induced cardiomyocytes of the present invention, to an individual.

For example, in some embodiments, the therapeutic method of the present invention comprises: i) generating induced cardiac progenitor cells or induced cardiomyocytes in vitro; and ii) introducing the induced cardiac progenitor cells or the induced cardiomyocytes into an individual in need thereof.

In addition, the present invention provides a method for reprogramming fibroblasts in vivo in cardiac tissues. The present method can be utilized to treat an individual. In some embodiments, the therapeutic method of the present invention comprises allowing an inducer or a composition containing a reprogramming factor(s) to come into contact with the fibroblasts of an individual in vivo. The contact comprises administration of the inducer or reprogramming composition of the present invention into a therapeutic site or a site close thereto, for example, into the heart or in the periphery thereof, in an individual body. The administration method is, for example, a method comprising inducing a catheter that has been inserted into the end of the artery to a site close to the diseased cardiac portion, and then injecting the inducer or composition comprising a reprogramming factor(s) of the present invention into the fibroid diseased tissues, through the tip of the catheter.

The therapeutic method of the present invention is useful to treat an individual suffering from cardiac or cardiovascular diseases or disorders, such as cardiovascular disease, aneurysm, angina, arrhythmia, atherosclerosis, cerebrovascular accidental disease (stroke), cardiovascular disease, congenital heart disease, congestive heart failure, myocarditis, coronary venous valve disease, scalability artery disease, diastolic dysfunction, endocarditis, hypertension, cardiomyopathy, hypertrophic cardiomyopathy, restrictive cardiomyopathy, coronary disease resulting in ischemic cardiomyopathy, mitral valve prolapse, myocardial infarction (heart attack), or venous thromboembolism.

The unit dosage form of an induced cardiomyocyte population, or a population of fibroblasts, induced cardiac progenitor cells or induced cardiomyocytes, may comprise approximately 10³ to approximately 10⁹ cells, for example, approximately 10³ to approximately 10⁴, approximately 10⁴ to approximately 10⁵, approximately 10⁵ to approximately 10⁶, approximately 10⁶ to approximately 10⁷, approximately 10⁷ to approximately 10⁸, or approximately 10⁸ to approximately 10⁹ cells.

### EXAMPLES

### [Example 1] Production of Mouse Embryonic Fibroblasts (MEF)

### (1) Production of Mouse Embryonic Fibroblasts (MEF)

Female ICR mice (CLEA Japan, Inc.) at 7 to 10 weeks after birth were mated with Mesp1-GFP transgenic mice (male) (Development 126, 3437-3447 (1999), “MesP1 is expressed in the heart precursor cells and required for the formation of a single heart tube”). The day on which fertilization was confirmed was defined as Day 0 of pregnancy, and on Day 12 after confirmation of the pregnancy, embryos were excised from the pregnant ICR mice. Then, the heart was excised from each embryo, and the excised heart was then imaged with fluorescence under an inverted microscope (IX71, Olympus). Thereafter, embryos emitting GFP fluorescence were selected.

From the selected embryos, four limbs, and solid organs such as ½ to ⅔ of head portion, lung, liver, kidney and intestinal tract were excised. The tissues of the remaining trunk of the body were washed with PBS (phosphate buffered saline)(−) (045-29795, WAKO), so that the blood cell components were fully removed. Thereafter, using sterilized surgical scissors, the tissues were sheared into as many as sections. To the sheared tissue sections, a solution comprising 0.25% Trypsin-EDTA (25200-072, Gibco) and PBS (−) (045-29795, WAKO) at a mixing ratio of 1:1 was added (15 mL/6 to 7 embryos), and the cells were then incubated while shaking at 37° C. for 15 minutes in a water bath. After that, 15 mL of a stock solution of FBS (Fetal Bovine Serum) (SV30014.03, Thermo Scientific) was added to the mixture, and was then fully suspended therein. The obtained suspension was centrifuged at 1500 rpm for 5 minutes at 4° C., and a supernatant was then removed.

The cell precipitate and suspension were re-suspended in 30 mL of a medium for MEF (10% FBS/DMEM/PSA) (Table 1), and the obtained re-suspension was inoculated in a 10-cm dish for tissue culture (172958, Thermo Scientific), so that the re-suspension corresponding to 2 or 3 embryos could be inoculated in the single 10-cm dish. The cells were cultured under conditions of 37° C./5% CO₂, and on the following day, the medium was exchanged with a fresh medium for MEF. Thereafter, medium exchange was continuously carried out every 3 or 4 days.

TABLE 1
Medium for MEF

| | | |
|---|---|---|
| FBS (Fetal Bovine Serum) (Thermo Scientific, SV30014.03) | 50 | mL |
| DMEM (WAKO, 044-29765) | 440 | mL |
| PSA | 5 | mL |
| Sodium Pyruvate (Sigma, S8636) | 5 | mL |
| GlutaMAX (Gibco, 35050-061) | 5 | mL |
| Non-essential amino acids solution 100x (Sigma, M7145) | 5 | mL |

### (2) Sorting of Mouse Embryonic Fibroblasts (MEF) by Flow Cytometry

When mouse embryonic fibroblasts (MEF) were induced to differentiate into cardiac progenitor cells, sorting was carried out by flow cytometry (FACS) as follows, and GFP (−) cells were used.

The medium was aspirated, and the cells were then washed with PBS (−). Thereafter, 2 mL of 0.25% Trypsin-EDTA was added to each dish, and the cells were then left at rest under conditions of 37° C./5% CO₂ for 5 minutes. After the floating of the cells in the culture medium had been confirmed, the reaction was neutralized with 8 mL of a medium for MEF (10% FBS/DMEM/PSA) (Table 1), and the cells were then recovered in a 15-mL tube (430791, Corning). The recovered cells were centrifuged under conditions of 1500 rpm/5 minutes/4° C. After aspiration of the supernatant, 350 μL of a solution for performing FACS (5% FBS/PBS) (Table 2) was added to the cells, and the cells were then fully suspended. This suspension was filtered using a 5-mL polystyrene round tube with cell strainer cap (REF 353335, FALCON), so as to obtain a sample for use in FACS. Using FACS (FACS AriaIII, Nippon Becton Dickinson Company, Ltd.), GFP-positive cells were separated from GFP-negative cells in the above-described sample, and the negative cells were used to induce differentiation into cardiac progenitor cells.

TABLE 2
Solution for performing FACS

| | | |
|---|---|---|
| FBS (Fetal Bovine Serum) (Thermo Scientific, SV30014.03) | 10 | mL |
| PBS (—) (WAKO, 045-29795) | 190 | mL |

### [Example 2] Production of Induced Cardiac Progenitor Cells and Cell Culture

Plat-E packaging cells were inoculated at a concentration of 3.6×10⁶ cells in a gelatin-coated 10-cm dish for tissue culture (172958, Thermo Scientific), and were then left at rest under conditions of 37° C./5% CO₂ (Day 1).

On the following day (Day 2), 27 μL of FuGENE 6 Transfection Reagent (E2691, Promega) was mixed into 300 μL of Opti-MEM (31985-070, Gibco). After the mixed solution had been left at rest for 5 minutes, 9000 ng of a retrovirus plasmid of pMx-Tbx6 (see Cell 142, 375-386, Aug. 6, 2010, “Direct Reprogramming of Fibroblasts into Functional Cardiomyocytes by Defined Factors”) was added to the mixed solution, followed by strong tapping, and thereafter, the obtained mixture was left at rest at room temperature for 15 minutes. The thus obtained solution was added dropwise to the Plat-E cells as a whole that had been prepared on the previous day (Day 1), and the dish was then left at rest under conditions of 37° C./5% CO₂ (transfection).

Twenty-four hours later (Day 3), the medium was exchanged with a Plat-E culture medium (DMEM/10% FBS/PSA) (Table 3), and it was then left at rest for further 24 hours under conditions of 37° C./5% CO₂.

TABLE 3
Medium for Plat-E

| | | |
|---|---|---|
| FBS (Fetal Bovine Serum) (Thermo Scientific, SV30014.03) | 50 | mL |
| DMEM (WAKO, 044-29765) | 440 | mL |
| PSA | 5 | mL |
| Sodium Pyruvate (Sigma, S8636) | 5 | mL |
| GlutaMAX (Gibco, 35050-061) | 5 | mL |
| Non-essential amino acids solution 100x (Sigma, M7145) | 5 | mL |

Meanwhile, the MEF of Example 1 was inoculated at a concentration of 0.5×10⁵ cells/well in a 12 Well Cell Culture Multiwell Plate (353043, FALCON), and was then cultured using a medium for MEF under conditions of 37° C./5% CO₂.

Forty-eight hours later (Day 4), each culture supernatant was filtrated through a Minisart filter with a pore size of 0.45 μm (17598, Sartorius Stedim Biotech), and was then recovered in a 50-mL tube (430829, Corning). Into 10 mL of the recovered supernatant, 4 μL of Polybrene Transfection Reagent (10 mg/mL) (#TR-1003-G, Millipore) was mixed. The obtained solution was defined as a retrovirus solution used for the Tbx6 gene.

The medium of the MEF of Example 1, which had been inoculated in the 12 Well Cell Culture Multiwell Plate on the previous day (Day 3), was aspirated, and the medium was then exchanged with a Tbx6 retrovirus solution, so that the cells were infected with the virus (infection).

On the following day of the gene introduction, the medium was exchanged with a medium for reprogramming of cardiac progenitor cells (Table 4), and the cells were then cultured under conditions of 37° C./5% CO₂. Thereafter, the medium was exchanged with a fresh medium every 3 or 4 days, while the cell culture was continued.

TABLE 4
Medium for reprogramming of cardiac progenitor cells

| | | |
|---|---|---|
| FBS (Fetal Bovine Serum) (Thermo Scientific, SV30014.03) | 50 | mL |
| DMEM (WAKO, 044-29765) | 440 | mL |
| PSA | 5 | mL |

### [Example 3] Production of Induced Cardiomyocytes Using Tbx6, SRF and Myocd, and Cell Culture

### (1) Method of Introducing Tbx6, SRF and Myocd Genes, Using Retrovirus

A virus solution was produced in the same manner as “Production of induced cardiac progenitor cells” of Example 2. In the present example, however, three genes were introduced into the cells. On Day 1, Plat-E cells were prepared in three 10-cm dishes for tissue culture by the same method as that applied in Example 2. On Day 2, 27 μL of FuGENE 6 Transfection Reagent (E2691, Promega) was mixed into 300 μL Opti-MEM (31985-070, Gibco). After the mixed solution had been left at rest for 5 minutes, 9000 ng of a retrovirus plasmid of each of pMx-Tbx6, pMx-SRF, and pMx-Myocd (see Cell 142, 375-386, Aug. 6, 2010, “Direct Reprogramming of Fibroblasts into Functional Cardiomyocytes by Defined Factors” with respect to their production technique) was added to the mixed solution, followed by strong tapping, and thereafter, the obtained mixture was left at rest at room temperature for 15 minutes. The thus obtained solution was added dropwise to the Plat-E cells as a whole that had been prepared on the previous day (Day 1), and the dishes were then left at rest under conditions of 37° C./5% CO₂ (transfection).

Twenty-four hours later (Day 3), the medium was exchanged with a Plat-E culture medium (DMEM/10% FBS/PSA) (Table 3), and it was then left at rest for further 24 hours under conditions of 37° C./5% CO₂. Forty-eight hours later (Day 4), each culture supernatant was filtrated through a Minisart filter with a pore size of 0.45 μm (17598, Sartorius Stedim Biotech), and was then recovered in a 50-mL tube (430829, Corning). Into 10 mL of the recovered supernatant, 4 μL of Polybrene Transfection Reagent (10 mg/mL) (#TR-1003-G, Millipore) was mixed. The obtained solution was defined as a retrovirus solution used for each gene (Tbx6, SRF, and Myocd).

On Day 3, a Matrigel solution (70.4 μg/mL) prepared by diluting 0.25 mL of Matrigel Growth Factor Reduced (354230, Corning) with 28.2 mL of DMEM was added in an amount of 0.25 mL to each well of a 12 Well Cell Culture Multiwell Plate (353043, FALCON), and it was then left at rest for 1 hour at 37° C., so that the plate was coated with Matrigel. After the removal of the solution, MEF (Example 1) was inoculated therein at a concentration of 0.5×10⁵ cells/well. Thereafter, the culture was continued using a medium for MEF (Table 1).

On Day 4, the medium for MEF was aspirated from the plate prepared on Day 3, and the medium was then exchanged with a retrovirus solution comprising Tbx6, SRF, and Myocd in equal amounts, so that the MEF was infected with the virus (infection).

### (2) Cell Culture Using Cardiomyocyte Induction Medium

On the day following the gene introduction (Day 5), the medium was exchanged with an FFV medium (Table 5), and the culture was then carried out under conditions of 37° C./5% CO₂. Thereafter, the medium was exchanged with a fresh medium every 3 or 4 days, while the cell culture was carried out.

TABLE 5
FFV Medium

| | | |
|---|---|---|
| StemPro(R) 34 SFM (Gibco, 10639-011) with Nutrient | 10 | mL |
| Supplement | | |
| GlutaMAX (Gibco, 35050-061) | 100 | μL |
| 5 mg/mL Ascrobic Acid (Sigma, A-4544) | 200 | μL |
| 5 ng/μL Recombinant Human VEGF165 (R & D Systems, | 10 | μL |
| 293-VE-050) | | |
| 10 ng/μL Recombinant Human FGF basic146 aa (R & D | 10 | μL |
| Systems, 233-FB-025) | | |
| 50 ng/μL Recombinant Human FGF10 (R & D Systems, | 5 | μL |
| 345-FG-025) | | |

### [Example 4] Induction of Cardiac Progenitor Cells from Mouse Fibroblasts

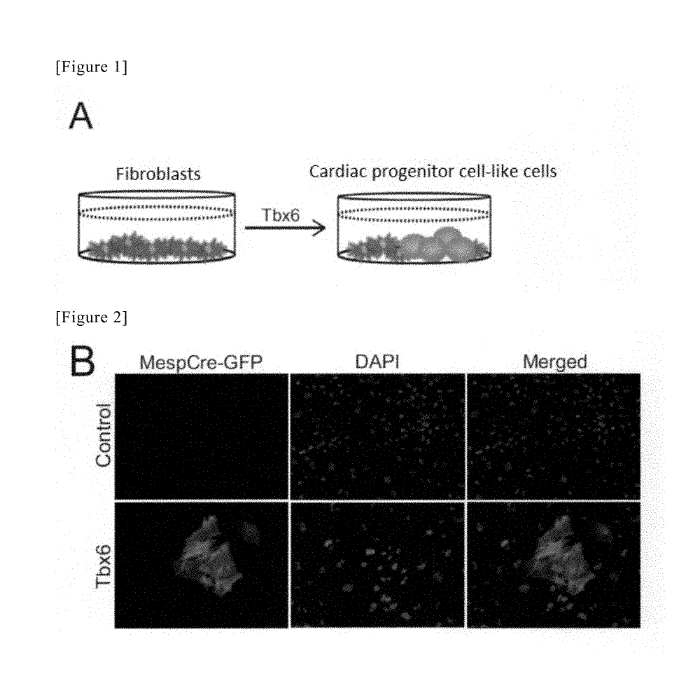

A Tbx6 gene was introduced into mouse fibroblasts (MEF), so that the MEF was induced to differentiate into cardiac progenitor cells (FIG. 1, Example 2).

Mesp1-Cre GFP flox mouse fibroblasts express GFP when expressing a cardiac progenitor cell-specific transcriptional factor Mesp1. A Tbx6 gene was introduced into the Mesp1-Cre GFP flox mouse fibroblasts (Example 2), and thereafter, it was confirmed that Mesp1-expressing GFP-positive cells formed colonies (FIG. 2).

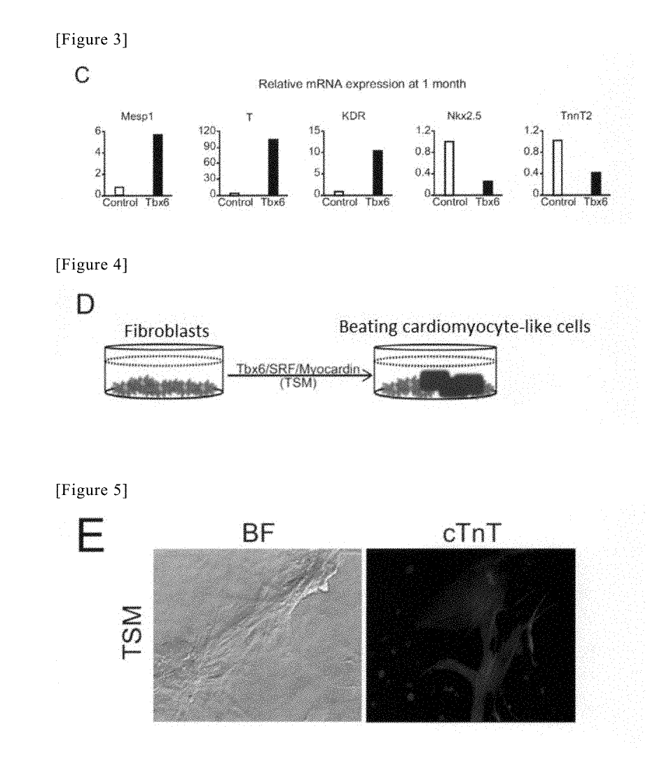

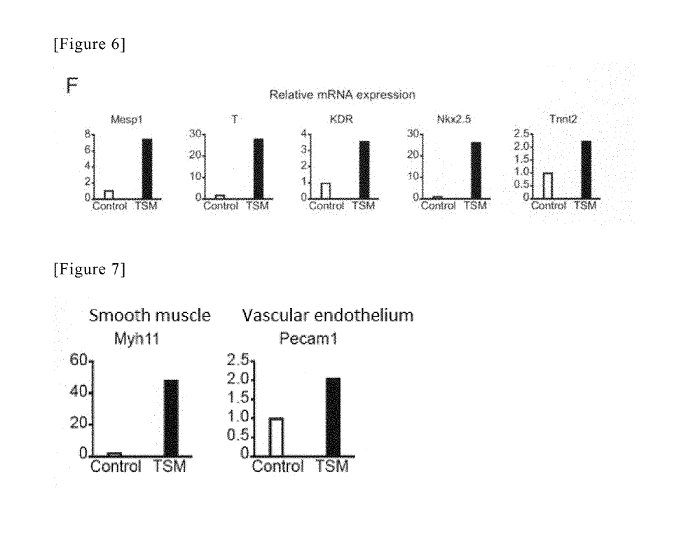

Subsequently, the mRNA expression of a differentiation marker gene was measured in the cells, into which the Tbx6 gene had been introduced. One month after the induction, the expression of cardiac progenitor cell-specific genes Mesp1 and T (brachyury), and further, the expression of KDR were maintained. On the other hand, the expression of Nkx2.5 or troponinT, which is expressed in differentiated cardiomyocytes, was not found (FIG. 3).

Examples 2 and 4 demonstrated that fibroblasts are induced to differentiate into cardiac progenitor cells by introducing Tbx6 into the fibroblasts.

### [Example 5] Induction of Cardiomyocytes from Mouse Fibroblasts

The Tbx6, SRF and Myocd genes were introduced into mouse fibroblasts (MEF), so that the MEF was induced to differentiate into cardiomyocytes (FIG. 4, Example 3).

The forms of the induced cardiomyocytes were changed, and the induced cardiomyocytes exhibited a striated structure (FIG. 5). In addition, the expression of troponinT (cTnT) as a structural protein of the cardiac muscle, or the expression of SM-MHC as a protein of smooth muscle cells, was found (FIG. 5).

The mRNA expression of the induced cells as a whole was examined. As a result, it was demonstrated that cardiomyocyte-specific genes that had not been induced by the single use of Tbx6, such as Nkx2.5 or troponinT (Tnnt2), were expressed, in addition to the induction of cardiac progenitor cell genes (Mesp1, T, and KDR).

Examples 3 and 5 demonstrated that fibroblasts are induced to differentiate into cardiomyocytes by introducing the Tbx6, SRF and Myocd genes into the fibroblasts.

### [Example 6] Induction of Smooth Muscle Cells and Vascular Endothelial Cells from Mouse Fibroblasts

The Tbx6, SRF, and Myocd genes were introduced into mouse fibroblasts (MEF) by the same method as that applied in Example 3.

On Day 14 after the gene introduction, immunostaining was carried out using an anti-Smooth muscle myosin heavy chain (myosin-11 or SMMHC) antibody.

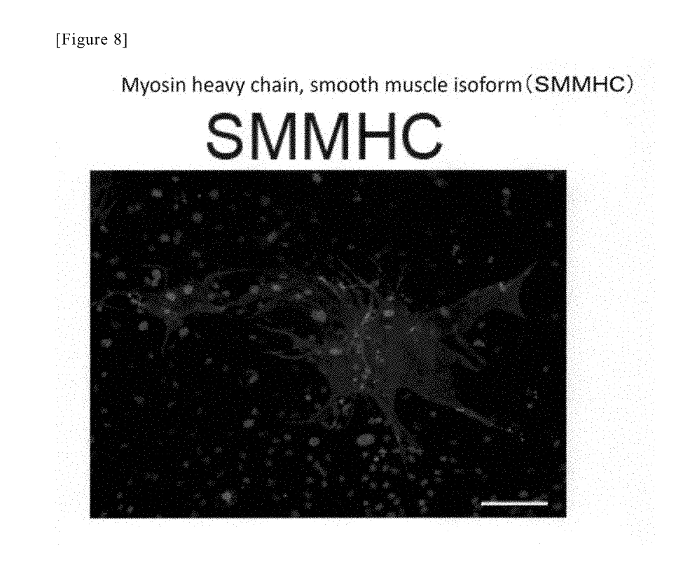

As a result, the cells, into which the Tbx6, SRF and Myocd genes had been introduced, exhibited positive to the aforementioned antibody. Thus, it is found that the cells expressed a myosin heavy chain that is a feature of the smooth muscle (FIG. 8).

Moreover, on Day 14 after the gene introduction, the expression levels of myosin heavy polypeptide 11 (Myh11) and Platelet/endothelial cell adhesion molecule 1 (Pecam1) were measured according to qRT-PCR. Myh11 is a protein specifically expressed in the smooth muscle, whereas Pecam1 is a protein specifically expressed in vascular endothelial cells.

As a result, it was confirmed that the expression of Myh11 and Pecam1 was induced in the cells into which the Tbx6, SRF and Myocd genes had been introduced (TSM) (FIG. 7).

The present example demonstrated that smooth muscle cells or vascular endothelial cells can be induced by introducing the Tbx6, SRF and Myocd genes into fibroblasts (MEF).

### INDUSTRIAL APPLICABILITY

According to the present invention, a method for directly producing cardiac progenitor cells from fibroblasts and a method for directly producing cardiomyocytes from fibroblasts are provided. In addition, the present invention can provide cardiac progenitor cells and cardiomyocytes, which are produced by the method of the present invention. Since the cardiac progenitor cells induced from fibroblasts according to the present invention have maintained the expression of a plurality of cardiac progenitor cell genes, the present invention can provide a method for producing cardiac progenitor cells, which is more stable than conventional methods, and cardiac progenitor cells. Since cardiac progenitor cells have proliferation ability, the cardiac progenitor cells produced by the present invention can be preferably applied to medical use.

Moreover, the cardiomyocytes induced by the present invention have been confirmed to beat, and further, the expression of a cardiac muscle-specific gene or the expression of a structural protein has been confirmed in the present cardiomyocytes. Therefore, the method for producing cardiac muscle of the present invention can provide functionally mature cardiomyocytes.

### SEQUENCE LISTING FREE TEXT

SEQ ID NO: 1: Nucleotide sequence of human Tbx6.

SEQ ID NO: 2: Amino acid sequence of human Tbx6.

SEQ ID NO: 3: Nucleotide sequence of mouse Tbx6.

SEQ ID NO: 4: Amino acid sequence of mouse Tbx6.

SEQ ID NO: 5: Nucleotide sequence of human SRF.

SEQ ID NO: 6: Amino acid sequence of human SRF.

SEQ ID NO: 7: Nucleotide sequence of mouse SRF.

SEQ ID NO: 8: Amino acid sequence of mouse SRF.

SEQ ID NO: 9: Nucleotide sequence of human Myocd.

SEQ ID NO: 10: Amino acid sequence of human Myocd.

SEQ ID NO: 11: Nucleotide sequence of mouse Myocd.

SEQ ID NO: 12: Amino acid sequence of mouse Myocd.

1. A method for producing cardiac progenitor cells, comprising a step of introducing a Tbx6 gene into fibroblasts.

2. A method for producing cardiomyocytes, comprising a step of introducing a Tbx6 gene, an SRF gene and a Myocardin gene into fibroblasts.

3. A cardiac progenitor cell derived from a fibroblast, comprising an exogenous Tbx6 gene.

4. A cardiomyocyte derived from a fibroblast, comprising an exogenous Tbx6 gene, an exogenous SRF gene and an exogenous Myocardin gene.

5. An inducer for inducing cardiac progenitor cells from fibroblasts, wherein the inducer comprises a Tbx6 gene.

6. An inducer for inducing cardiomyocytes from fibroblasts, wherein the inducer comprises a Tbx6 gene, an SRF gene and a Myocd gene.

7. An inducer for inducing smooth muscle cells from fibroblasts, wherein the inducer comprises a Tbx6 gene, an SRF gene and a Myocd gene.

8. An inducer for inducing vascular endothelial cells from fibroblasts, wherein the inducer comprises a Tbx6 gene, an SRF gene and a Myocd gene.
